(19) United States
(12) Patent Application Publication
(10) Pub. No.: US 2005/0192299 A1
(43) Pub. Date: Sep. 1, 2005

(54) Method of treating or preventing hepatitis B virus

(76) Inventors: Yung-Chi Cheng, Woodbridge, CT (US); Chung K. Chu, Athens, GA (US); Hea O. Kim, Seoul (KR); Kirupathevy Shanmuganathan, San Antonio, TX (US)

Correspondence Address: JONES DAY, 222 EAST 41ST ST, NEW YORK, NY, 10017, US

(21) Appl. No.: 10/656,889

(22) Filed: Sep. 4, 2003

Related U.S. Application Data
(63) Continuation of application No. 08/463,960, filed on Jun. 5, 1995, now abandoned.
Application No. 08/463,960 is a division of application No. 08/331,164, filed on Oct. 28, 1994, now abandoned.
Application No. 08/331,164 is a continuation of application No. 07/937,845, filed on Oct. 19, 1992, now abandoned.

(30) Foreign Application Priority Data
Apr. 16, 1992 (WO) PCT/US92/03144

(51) Int. Cl. A61K 31/513; A61K 38/21; A61K 31/7076

(52) U.S. Cl. 514/269; 424/85.7; 514/47

(57) ABSTRACT

Anti-hepatitis B virus compounds (−)3′-thia-2′,3′-dideoxycytidine, (−)5-fluoro-3′-thia-2′,3′-dideoxycytidine, (±) β-dioxolane cytosine and (−)-L-B-dioxolane cytosine. A method of treating a patient suffering from hepatitis B virus or preventing hepatitis B virus infection comprising administering to the patient an effective amount of an active compound selected from the group consisting of (a) (−)3′-thia-2′,3′-dideoxycytidine, (b) (±)3′-thia-2′3′-dideoxycytidine, (c) (−)5-fluoro-3′-thia-2′,3′-dideoxcytidine; (d) (±) 5-fluoro-3′-thia-2′3′-dideoxycytidine, (e)(±) β-dioxolane-cytosine and (f) (−)-L-β-dioxolane cytosine, or a salt or an ester thereof, either alone or in admixture within a diluent.

### CROSS-REFERENCES TO RELATED APPLICATIONS

This is a continuation-in-part application of application Ser. No. 07/785,545, filed on Oct. 31, 1991, which in turn is a continuation-in-part application of Ser. No. 07/718,806, filed Jun. 21, 1991, which in turn is a continuation-in-part application of application Ser. No. 07/686,617, filed Apr. 17, 1991, now abandoned.

### GOVERNMENT RIGHTS

This invention was made with United States Government support under Grant CA-44358 from the National Cancer Institute (NIH). Accordingly, the United States Government has certain rights in this invention.

### BACKGROUND OF THE INVENTION

1. Field of the Invention

The present invention concerns (−)3′-thia-2′3′-dideoxycytidine and (−)5-fluoro-3′-thia-2′,3′-dideoxycytidine, a method for preparing the same and the use of the same or (±)3′-thia-2′,3′-dideoxycytidine or (±)5-fluoro-3′-thia-2′,3′-dideoxycytidine in a method for treating patients having hepatitis B virus or to prevent hepatitis B virus infection.

The present invention also relates to dioxolane-cytosine and particularly (±)-L-β-dioxolane-cytosine and its use in a method for treating patients having hepatitis B virus or to prevent hepatitis B virus infection.

2. Background Information

Hepatitis B virus (HBV) causes acute or chronic hepatitis which affects nearly 300 million people worldwide (Ayoola, E. A., Balayan. M. S., Deinhardt, F. Gust, I. Kureshi, A. W., Maynard, J. E., Nayak, N. C., Bordley, D. W., Ferguson, M. Melnick, J., Purcell, R. H. and Zuckerman, A. J., Bull. World Health. Org., 66: 443-455, 1988). Chronic infection with HBV has been associated with a high risk for the development of primary hepatocellular carcinoma (Beasley, R. P., Hwang, L. Y., Lin, C. C., and Chien, C. S., “Hepatocellular Carcinoma and Hepatitis B Virus,” Lancet ii: 1129-1133, 1981; Di Bisceglei, A. M., Rustgi, V. K., Hoofnagle, J. H., Dusheik, G. M., and Lotze, M. T., “Hepatocellular Carcinoma,” Ann. Intern. Med. 108: 390-401, 1988).

Effective antiviral therapy against HBV infection has not been fully developed. It has been hampered by the extremely narrow host-range and limited access to experimental culture systems. Lately, Hepadnaviruses have been propagated in tissue culture (Sureau, C., Romet-Lomonne, J. L., Mullins, J. I., and Essex, M., “Production of Hepatitis B Virus by a Differentiated Human Hepatoma Cell Line after Tranfection with Cloned Circular HBV DNA,” Cell 47:37-47; 1986; Chang, C., Jeng, K. S., Hu, C. P., Lo, S. J., Su, T. S., Ting, L. P., Chou, C. K., Han, S. H., Pfaff, E., Salfeld, J., and Schaller, H., “Production of Hepatitis B Virus in Vitro by Transient Expression of Cloned HBV DNA in a Hepatoma Cell Line,” EMBO J. 6: 675-680, 1987; Tsurimoto, T., Fujiyama, A., and Matsubara, K., “Stable Expression and Replication of Hepatitis B Virus Genome in an Integrated State in a Human Hepatoma Cell Line Transfected with the Cloned Viral, DNA,” Proc. Natl. Acad. Sci. USA, 84: 444-448, 1987; and Sells, M. A., Chen, M. L., and Acs, G., “Production of Hepatitis B Virus Particles in Hep G2 Cells Transfected with Cloned Hepatitis B Virus DNA,” Proc. Natl. Sci. USA, 84: 1005-1009, 1987, making it possible to study various aspects of the viral life cycle and screening for antiviral drugs.

Hepadnaviruses replicate through a ribonucleic acid (RNA) template that requires reverse transcriptase activity (Ganem, D., and Varmus, H. E., “The Molecular biology of the hepatitis B viruses,” Ann. Rev. Biochem. 56: 651-693, 1987). The rationale for a chemotherapeutic treatment for hepatitis B is the inhibition of the viral DNA polymerase. HBV DNA polymerase has a common evolutionary origin with the reverse transcriptase from retroviruses (Miller, R. H., and Robinson, W. S., “Common Evolutionary Origin of Hepatitis B Virus and Retroviruses,” Proc. Natl. Acad. Sci. USA, 83: 2531-2535, 1986).

Inhibitors for reverse transcriptase of oncogenic RNA viruses suppress the polymerase from HBV (Matthes, E., Langen, P., von Janta-Lipinski, M., Will, H. Schroder, H. C., Merz, H., Weiler, B. E., and Muller, W. E. G., “Potent Inhibition of Hepatitis B Virus Production in Vitro by Modified Pyrimidine Nucleosides,” Antimicrobial Agents and Chemotherapy, 34: 1986-1990, 1990; Lee, B., Luo, W., Suzuk, S., Robins. M. J., and Tyrrell, D. L. J., “In Vitro and in Vivo Comparison of the Abilities of Purine and Pyrimidine 2′,3′-Dideoxynucleosides to Inhibit Duck Hepadnavirus,” Antimicrobial Agents and Chemotherapy, 33: 336-339, 1989). Several 2′3′-dideoxynucleoside analogs have been used as potential antiretroviral agents. 2′3-Dideoxycytidine (ddC) has been shown to be the most potent inhibitor of HIV replication in cell culture (Mitsuya, H., Yarchoan, R., and Broder, S., “Molecular Targets for AIDS Therapy,” Science, 249: 1533-1544, 1990). ddC was also shown to have potent antiviral activity against duck hepatitis B virus both in vitro (Lee, B., Luo, W., Suzuk, S., Robins, M. J., and Tyrrell, D. L. J., “In Vitro and in Vivo Comparison of the Abilities of Purine and Pyrimidine 2′,3′-dideoxynucleosides to Inhibit Duck Hepadnavirus,” Antimicrobial Agents and Chemotherapy, 33: 336-339, 1989) and in vitro (Kassianides, L., Hoofnagle, J. H., Miller, R. H., Doo, E., Ford, H., Broder, S., and Mitsuya, H., “Inhibition of Duck Hepatitis B Virus Replication by 2′,3′-Dideoxycytidine,” Gastroenterology, 97: 1275-1280, 1989).

Studies have indicated that elimination of mitochondria DNA by treatment of ddC is related to delayed cytotoxicity and possibly resulted in peripheral neuropathy observed in clinics (Chen, C. H., Cheng, Y. C., “Delayed Cytotoxicity and Selective Loss of Mitochondria DNA in Cells Treated with the Anti-Human Immunodeficiency Virus Compound 2′,3′-Dideoxycytidine,” J. Biol. Chem., 264: 11934-11937, 1989).

EP 382 526, the entire contents of which are incorporated by reference herein, concerns substituted 1,3-oxathiolanes which are said to have antiviral properties. However, only retroviruses are specifically discussed and the only specific viral disorders described are human immunodeficiency virus (HIV) (AIDS), AIDS related conditions such as AIDS-related complex (ARC), persistent lymphadenopathy (PGL), AIDS-related neurological conditions (such as dementia), Kaposi's sarcoma and thrombocytopenia purpurea.

The use of BCH-189 ((±)-2′,3′-dideoxy-3′-thiacytidine) and dioxolane-T as anti HIV agents are discussed in Chu et al, J. Org. Chem., 56, 6503-6505, (1991); Jeong et al, Tetrahedron Letters, 33, 595-598, (1992); Chu et al, Tetrahedron Letters, 32, 3791-37.94 (1991) and B. Belleau et al V International Conference on AIDS, Montreal, Canada, Jun. 4-9 (1989), paper No. T.C.O.1.

BCH-189 was reported as a racemic mixture in J. A. Coates et al, Antimicrob. Acents Chemother. 36, 202 (1992) and W.-B. Choi et al, J. Am. Chem. Soc., 11, 9377 (1991).

In summary, there is currently no effective treatment for human hepatitis B virus infections. Antiviral therapy and a variety of experimental drugs have had no clear benefit and some may be harmful. Recently, a variety of 2′,3′-dideoxyguanosine analogues have been asserted to have anti-HBV activity in vitro, yet none have reached clinical usefulness. In general, guanosine analogues of this type may interfere with critical enzymes in humans.

NOMENCLATURE

| | |
|---|---|
| 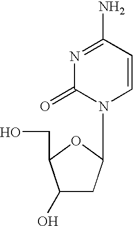 | 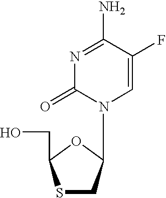 |
| <1> dC | <5> FSddc |
| deoxycytidine | (31 )5-fluoro-3′-thia-2′,3′- |
| | dideoxycytidine |
| 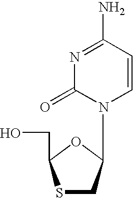 | 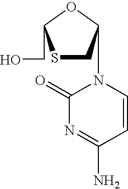 |
| <2> + SddC | <3> -SddC |
| (+)3′-thia-2′,3′-dideoxycytidine | (−)3′-thia-2′,3′-dideoxycytidine |
| 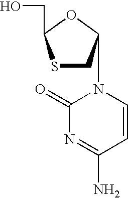 | 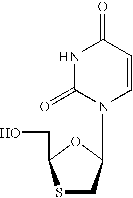 |
| <4> α SddC | (α) SddU |
| α 3′-thia-2′,3′-dideoxycytidine | (+)3′-thia-2′,3′-dideoxyuridine |
| 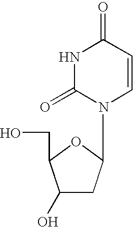 | 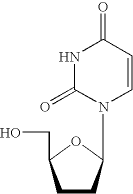 |
| dU (2′-deoxyuridine) | ddU (2′,3′-dideoxyuridine) |
| 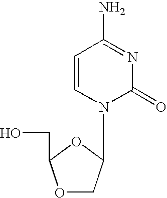 | 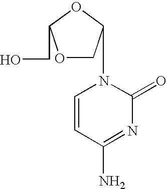 |
| (+) -D-B-dioxolane-cytosine | (−) -L-B-dioxolane-cytosine |
| ((+) OddC) | ((−) OddC) |

SddU: deaminate form of SddC (3′-thia-2′,3′-dideoxycytidine)
5-FSddC: 5-fluoro-3′-thia-2′,3′-dideoxycytidine
SddC: 3′-thia-2′,3′-dideoxycytidine
5-MeSddC: 5-methyl-3′-thia-2′,3′-dideoxycytidine
5-ClSddC: 5-chloro-3′-thia-2′,3′-dideoxycytidine
5-BrSddC: 5-bromo-3′-thia-2′,3′-dideoxycytidine
5-ISddC: 5-iodo-3′-thia-2′,3′-dideoxycytidine
(+) OddC: (+)-D-B-dioxolane-cytosine or (+)D-B-dioxolane-C
(−) OddC: (−)-L-B-dioxolane-cytosine or (−)L-B-dioxolane-C

Unless indicated to the contrary, whenever 3′-thia-2′,3′-dideoxycytidine without a plus or minus sign before it is stated herein, it is understood that such means (±)3′-thia-2′,3′-dideoxycytidine and whenever 5-fluoro-3′-thia-2′,3′-dideoxycytidine without a plus or minus sign before it is stated herein, it is understood that such means (±)5-fluoro-3′-thia-2′,3′-dideoxycytidine. Furthermore, unless indicated to the contrary, whenever β-dioxolane-cytosine without a plus or minus sign before it is stated herein, it is understood that such means (±)β-dioxolane-cytosine.

### SUMMARY OF THE INVENTION

An object of the present invention is to provide (−)3′-thia-2′,3′-dideoxycytidine and (−)5-fluoro-3′-thia-2′,3′-dideoxycytidine and a method of preparing the same.

A further object of the present invention is to provide β-dioxolane cytosine and particularly (−)-L-8-dioxolane-cytosine.

It is another object of the present invention is to treat patients suffering with the hepatitis B virus or to prevent hepatitis B virus infection in a patient.

The above stated objects, and other objects, aims and advantages are provided by the present invention.

The present invention concerns (−)31-thia-2′,3′-dideoxycytidine of the following formula:

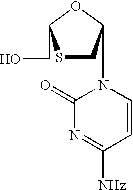

The present invention also relates to (−)5-fluoro-3′-thia-2′,3′-dideoxycytidine of the following formula:

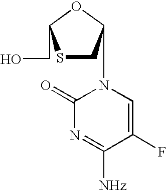

The instant invention is also directed to a method of separating (−)3′-thia-2′,3′-dideoxycytidine from (±)3′-thia-2′,3′-dideoxycytidine. The method comprises contacting (±)3′-thia-2′,3′-dideoxycytidine with deoxycytidine deaminase, subjecting the resultant reaction mixture to column chromatography, for example, HPLC, and separating out (−)3′-thia-2′,3′-dideoxycytidine. The above method is also applicable for separating (−)5-fluoro-3′-thia-2′,3′-dideoxycytidine from (±)5-fluoro-3′-thia-2′,3′-dideoxycytidine.

The present invention also concerns a method of treating hepatitis B virus infection or preventing hepatitis B virus infection in a patient, e.g., a mammal, e.g., a human comprising administering to the patient an effective amount of a substituted-1,3-oxathiolane compound selected from the group consisting of (−)3′-thia-2′,3′-dideoxycytidine, (−)3′-thia-2′,3′-dideoxycytidine, (−)5-fluoro-3′-thia-2′,3′-dideoxycytidine and (−)5-fluoro-3′-thia-2′,3′-dideoxycytidine, preferably (−)3′-thia-2′,3′-dideoxycytidine or (−)5-fluoro-3′-thia-2′,3′-dideoxycytidine, or a salt or ester thereof, either alone or in admixture with a pharmaceutically acceptable carrier.

The present invention also concerns β-dioxolane-cytosine and particularly (−)-L-β-dioxolane-cytosine of the formula

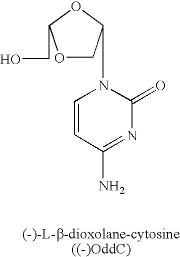

The present invention also concerns a method of treating hepatitis B virus infection or preventing hepatitis B virus infection in a patient, e.g., a mammal, e.g., a human comprising administering to the patient an effective amount of a β-dioxolane-cytosine, particularly (−)-L-β-dioxolane cytosine, or a salt or ester thereof, either alone or in admixture with a pharmaceutically acceptable carrier.

### BRIEF DESCRIPTION OF THE DRAWINGS

FIG. 1 depicts a Southern blot analysis of the comparative potency of deoxycytidine analogs as inhibitors of HBV 2.2.15 cells which were incubated with the various concentrations of drugs for 12 days. Media were harvested. Virions were precipitated with PEG. Nucleic acids were extracted from PEG precipitates and analyzed by Southern blot analysis. RC: Relaxed circular HBV DNAs. SS: single stranded HBV DNAs. D4C: 2′,3′-didehydro-2′,3′-dideoxycytidine. 3′-FddC: 3′-fluoro-2′,3′-dideoxycytidine. SddC: 3′-thia-2′,3′-dideoxycytidine. 5-FSddc: 5-fluoro-3′-thia-2′,3′-dideoxycytidine; ddC: 2′,3′ dideoxycytidine.

FIG. 2 is a Southern analysis of intracellular HBV DNAs. 2.2.15 cells were untreated (lanes 1,10), treated with 5-fluoro-3′-thia-2′,3′-dideoxycytidine. (lanes 2,3,4,5, and 11) and 3′-thia-2′,3′-dideoxycytidine (lanes 6,7,8,9,and 12) for 12 days. Total cellular DNAs were extracted as described in hereinbelow. DNAs were digested with Hind III and electrophoresed in 0.8% agarose gel, transferred to Hybond-N membrane and hybridized with ³²P-labeled HBV probe. Each lane represents 20 μg total cellular DNAs. Lanes 10, 11, 12: DNAs from cells untreated (lane 10) or treated with 2 μm 5-fluoro-3′-thia-2′,3-dideoxycytidine (lane 11) and 3′-thia 2′,3′-dideoxycytidine (lane 12) for 12 days and further incubation in the absence of drugs for 12 more days. RC: Relaxed circular episomal HBV DNAs; I: Integrated HBV DNAs.

FIG. 3 is a Southern blot depicting the reversibility of 5-fluoro-3′-thia-2′,3-dideoxycytidine and 3′-thia-2′,3′-dideoxycytidine. 2.2.15 cells untreated or treated with 2 μM of 5-fluoro-3′-thia-2′,3-dideoxycytidine and 3′-thia-2′,3′-dideoxycytidine for 12 days were incubated with drug-free medium for 6 or 12 more days. HBV specific DNAs in the medium were analyzed as described in FIG. 1. RC: Relaxed circular HBV DNAs. SS: Single stranded HBV DNAs.

FIG. 4 is a Northern blot analysis of RNAs. Total RNAs were extracted from 2.2.15 cells untreated (lane 1) or treated with 2.0 μm 5-fluoro-3′-thia-2′,3′-dideoxycytidine (lane 2) and 3′-thia-2′,3′-dideoxycytidine (lane 3) for 12 days. Each lane represents 20 μg total RNAs.

FIG. 5 is a Southern blot depicting the comparative potency of various analogs of 3′-thia-2′,3′-dideoxycytidine as inhibitors of HBV replication. 2.2.15 cells were treated with various analogues at 1.0 μM for 12 days. Media were analyzed for the presence of HBV DNAs as described in FIG. 1.

FIG. 6A depicts a EPLC profile of a mixture of (±)3′-thia-2′,3′-dideoxycytidine before a deamination.

FIG. 6B depicts a HPLC profile 16 hours after deamination of the mixture.

FIG. 7A depicts a HPLC profile of a control having only (+)3′-thia-2′,3′-dideoxycytidine before deamination.

FIG. 7B depicts a HPLC profile of a UV spectrum 16 hours after deamination of (+)3′-thia-2′,3′-dideoxycytidine.

FIG. 8A depicts a HPLC profile of (−)3′-thia-2′,3′-dideoxycytidine before a deamination.

FIG. 8B depicts a HPLC profile of (−)3′-thia-2′,3′-dideoxycytidine 16 hours after a deamination.

FIG. 9A depicts a HPLC profile of a-SddC before a deamination.

FIG. 9B depicts a HPLC profile of a-SddC 16 hours after a deamination.

FIG. 10 is a UV spectrum for 3′-thia-2′,3′-dideoxycytidine.

FIG. 11 is a UV spectrum for 3′-thia-2′,3′-dideoxyuridine.

FIGS. 12A and FIG. 12B each depict a Southern analysis of intracellular HBV DNA wherein 2.2.15 cells are untreated (control) or treated.

FIG. 13 is a Southern blot analysis of the comparative potency of several compounds as inhibitors of intracellular HBV DNA after a one week incubation with HBV 2.2.15 cells.

### DETAILED DESCRIPTION OF THE INVENTION

The rationale for a chemotherapeutic treatment for hepatitis B virus is the inhibition of the viral DNA polymerase. Many nucleoside analogs have been tested both in a tissue culture system and an animal model with varying success (Matthes, E., Langen, P., von Janta-Lipinski, M., Will, H. Schroder, H. C., Merz, H., Weiler, B. E., and Muller, W. E. G., “Potent Inhibition of Hepatitis B Virus Production in Vitro by Modified Pyrimidine Nucleosides,” Antimicrobial Agents and Chemotherapy, 34: 1986-1990, 1990; Lee, B., Luo, W., Suzuk, S., Robins, M. J., and Tyrrell, D. L. J., “In Vitro and in Vivo Comparison of the Abilities of Purine and Pyrimidine 2′,3′-Dideoxynucleosides to Inhibit Duck Hepadnavirus,” Antimicrobial Agents and Chemotherapy, 33: 336-339, 1989; Kassianides, L., Hoofnagle, J. H., Miller, R. H., Doo, E., Ford, H., Broder, S., and Mitsuya, H., “Inhibition of Duck Hepatitis B Virus Replication by 2′,3′-Dideoxycytidine,” Gastroenterology, 97: 1275-1280, 1989; Price, P. M., Banerjee, R., and Acs, G., “Inhibition of the Replication of Hepatitis B Virus by the Carbocyclic Analogue of 2′-Deoxyguanosine,” Proc. Natl. Acad. Sci., USA 86: 8541-8544, 1989; Yokota, T., Konno, K., Chonan, E., Mochizuki, S., Kojima, K., Shigeta, S., and de Clercq, E., “Comparative Activities of Several Nucleoside Analogues Against Duck Hepatitis B Virus in Vitro,” Antimicrobial Agents and Chemotherapy, 34: 1326-1330, 1990; and Ueda, K., Tsurimoto, T., Nagahata, T., Chisaka, O., and Matsubara, K., “An in Vitro System for Screening Antihepatitis B Drugs,” Virology 169: 213-216, 1989).

Applicants discovered that 3′-thia-2′,3′-dideoxycytidine and its 5-fluoro analog were potent compounds against HBV replication. It was further discovered that with respect to the anti-HBV effects of (±)3′-thia-2′,3′-dideoxycytidine and its racemic forms, namely (±)3′-thia-2′,3′-dideoxycytidine and (−)3′-thia-2′,3′-dideoxycytidine, that (−)3′-thia-2′,3′-dideoxycytidine is the primary active form responsible for the anti-HBV effect and the (±)3′-thia-2′,3′-dideoxycytidine is the major component causing the cytotoxicity at the concentration wherein the anti-HBV effect was observed.

In contrast to the effectiveness in inhibiting HBV replication, 3′-thia-2′,3′-dideoxycytidine and 5-fluoro-3′-thia-2′,3-dideoxycytidine were not found to affect the integrated HBV DNAs. Since the RNA replicative intermediates are being transcribed from the integrated DNA, it is not surprising that HBV specific transcripts were not affected by drug treatment. Thus, interruption of drug treatment resulted in a return of HBV virus to both intra- and extracellular populations.

Without wishing to be bound by any particular operability, the mechanism of action of 3′-thia-2′,3′-dideoxycytidine is probably (1) inhibition of viral DNA polymerase and/or (2) chain termination due to incorporation into elongated DNA strand. 3′-Thia-2′,3′-dideoxyuridine analogs were found not to be active against HBV replication. There was concern that cytidine analogs can be deaminated intracellularly to inactive uracil analog. The facts that 3′-thia-2′,3′-dideoxycytidine is very potent against HBV replication in 2.2.15 cells and that the anti-HBV activity is not enhanced by cyd/dcyd deaminase inhibitor suggests that catabolic inactivation by deaminase may not be important.

The present invention concerns a method involving the administration of one or more of (−)3′-thia-2′,3′-dideoxycytidine, (±)3′-thia-2′,3′-dideoxycytidine, (−)5-fluoro-3′-thia-2′,3-dideoxycytidine or (±)5-fluoro-3′-thia-2′,3′-dideoxycytidine (referred to herein as “the compounds of formula (I)”) or a salt or ester thereof, alone or in admixture with a pharmaceutically acceptable carrier in order to treat patients suffering from hepatitis B virus or to prevent hepatitis B virus infection.

Formula (I) includes the following: 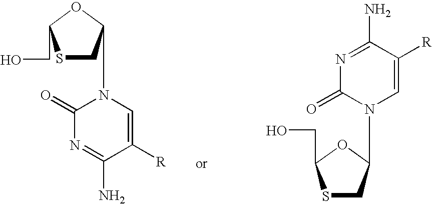
wherein R is selected from the group consisting of H and F.

As referred to herein, formula (I) refers to (−)3′-thia-2′,3′-dideoxycytidine, (±)3′-thia-2′,3′-dideoxycytidine, (−)5-fluoro-3′-thia-2′,3′-dideoxycytidine or (±)5-fluoro-3′-thia-2′,3′-dideoxycytidine or combinations thereof.

The present invention also concerns a method involving the administration of β-dioxolane-cytosine and particularly (−)-L-β-dioxolanecytosine of the formula 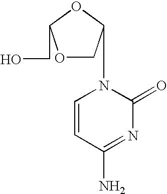
or a salt or ester thereof, alone or in admixture with a pharmaceutically acceptable carrier in order to treat patients suffering from hepatitis B virus or to prevent hepatitis B virus infection. β-dioxolane-cytosine or (−)-L-β-dioxolane-cytosine are hereinafter referred to as the compounds of formula (I′).

Preferred esters of the compounds for use in the invention of formula (I) include the compounds in which H of HOCH₂ is replaced by 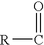
in which the non-carbonyl moiety R of the ester grouping is selected from hydrogen, straight or branched chain alkyl (e.g., methyl, ethyl, n-propyl, t-butyl, n-butyl), alkoxyalkyl (e.g., methoxymethyl), aralkyl (e.g., benzyl), aryloxyalkyl (e.g., phenoxymethyl), aryl (e.g., phenyl optionally substituted by halogen,. C₁₋₄ alkyl or C₁₋₄ alkoxy); substituted dihydro pyridinyl (e.g., N-methyldihydro pyridinyl); sulphonate esters such as alkyl or arakylsulphonyl (e.g., methanesulphonyl); sulphate esters; amino acid esters (e.g., L-valyl or L-isoleucyl) and mono-, di- or tri-phosphate esters.

Also included within the scope of such esters are esters derived from polyfunctional acids such as carboxylic acids containing more than one carboxyl group, for example, dicarboxylic acids HO₂C(CH₂)ₙCO₂H where n is an integer of 1 to 10 (for example, succinic acid) or phosphoric acids. Methods for preparing such esters are well known. See, for example, Hahn et al., “Nucleotide Dimers as Anti Human Immunodeficiency Virus Agents,” Nucleotide Analogues, pp.156-159 (1989) and Busso et al., “Nucleotide Dimers Suppress HIV Expression in Vitro,” AIDS Research and Human Retroviruses, 4(6), pp.449-455 (1988). Where esters are derived from such acids, each acidic group is preferable esterified by a compound for use in the invention or other nucleosides or analogues and derivatives thereof to provide esters of the formula (II) 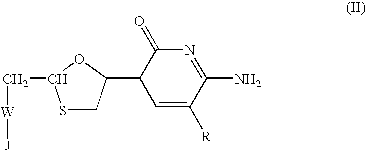
where R is H or F, W is 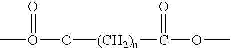
and n is an integer of 1 to 10 or 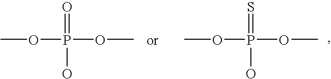
J is any nucleoside or nucleoside analog or derivative thereof. Preferred nucleosides and nucleoside analogues are 3′-azido-2′-3′-dideoxythymidine, 2′,3′-dideoxycytidine, 2′,3′-dideoxyadenosine, 2′,3′-dideoxyinosine, 2′,3′-dideoxythymidine, 2′,3′-dideoxy-2′,3′-didehydro-thymidine, and 2′,3′-dideoxy-2′,3′-didehydroxytidine and ribavirin.

With regard to the above described esters, unless otherwise specified, any alkyl-moiety present advantageously contains 1 to 16 carbon atoms, preferably 1 to 4 carbon atoms and could contain one or more double bonds. Any aryl moiety present in such esters advantageously comprises a phenyl group.

In particular, the esters may be a C₁₋₁₆ alkyl ester, an unsubstituted benzoyl ester r a benzoyl ester substituted by at least one halogen (bromine, chlorine, fluorine or iodine), saturated or unsaturated C_{1- 6} alkyl, saturated or unsaturated C₁₋₆ alkoxy, nitro or trifluoromethyl groups.

Pharmaceutically acceptable salts of the compounds of formula (I) or formula (I′) include those derived from pharmaceutically acceptable inorganic acids and bases. Examples of suitable acids include hydrochloric, hydrobromic, sulfuric, nitric, perchloric, fumaric, maleic, phosphoric, glycollic, lactic, salicylic, succinic, toluene-p-sulfonic, tartaric, acetic, citric, methanesulfonic, formic, benzoic, malonic, naphthalene-2-sulfonic and benzenesulfonic acids. Other acids such as oxalic, while not in themselves pharmaceutically acceptable, may be useful in the preparation of salts useful as intermediates in obtaining the compounds of formula (I) or formula (I′) and their pharmaceutically acceptable acid addition salts.

Salts derived from appropriate bases include alkali metal (e.g., sodium), alkaline earth metal (e.g., magnesium), ammonium and NR₄+ (where R is C₁₋₄ alkyl) salts.

The amount of the compound of formula. (I) or formula (I′) for use in the present invention will vary not only with the particular compound selected, but also with the route of administration, the nature of the condition being treated and the age and condition of the patient and will be ultimately determined by the discretion of the attendant, physician or veterinarian. In general, however a suitable dose will be in the range from about 1 to about 100 mg/kg of body weight per day, such as 2 to about 50 mg per kilogram body weight of the recipient per day, preferably in the range of 2 to 10 mg/kg/day.

The desired dose may conveniently be presented in a single dose or as divided doses administered at appropriate intervals, for example, at two, three, four or more sub-doses per day.

The compound of formula (I) or formula (I′) is conveniently administered in unit dosage; for example, containing 0.5 to 50 mg, conveniently 20 to 1000 mg most conveniently 50 to 700 mg, of active ingredient (compound of formula (I) or formula (I′)) per unit dosage form.

Ideally, the active ingredient should be administered to achieve peak plasma concentrations of the active compound of from about 1 to 75 μM, preferably about 2 to 50 μM, most preferably about 3 to about 30 μM. This may be achieved, for example, by the intravenous injection of 0.1 to 5% solution of the active ingredient, optionally in saline, or administered as a bolus containing about 0.1 to 50 mg/kg of the active ingredient.

While it is possible that, for use in therapy, the compound of formula (I) or formula (I′) may be, administered as the raw chemical, it is preferable to present the active ingredient as a pharmaceutical formulation.

The invention thus further provides for the use of a pharmaceutical formulation comprising a compound of formula (I) or formula (I′) or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient therefor.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

The compounds of formula (I) or formula (I′) may also be formulated for parental administration (e.g., by injection, for example, bolus injection or continuous infusion) and may be presented in unit dose form in ampoules, pre-filled syringes, small volume infusion or in multi-dose containers with an added preservative. The compositions may take such forms as suspensions, 'solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds according to formula (I) or formula (I′) may be formulated as ointments, creams or lotions, or as a transdermal patch. Ointments and creams may, for example, be formulated with an aqueous or oily base with the addition of suitable thickening and/or gelling agents. Lotions may be formulated with ah aqueous or oily base and will in general also contain one or more emulsifying agents, stabilizing agents, suspending agents, thickening agents, or coloring agents.

Formulations suitable for topical administration in the mouth include lozenges comprising an active ingredient in a flavored base, usually sucrose and acacia or tragacanth; pastilles comprising the active ingredient in a suitable liquid carrier.

Pharmaceutical formulations suitable for rectal administration, wherein the carrier is a solid, are most preferably represented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

Compounds of formula (I) or formula (I′) or formulations containing the same can also be applied on condoms (on the inner surface thereof, outer surface thereof or both of said surfaces) to prevent the transmission of HBV during intercourse.

For intra-nasal administration, the compounds of formula (I) may be used as a liquid spray or dispersible powder or in the form of drops.

Drops may be formulated with an aqueous or non-aqueous base comprising one or more dispersing agents, solubilizing agents or suspending agents. Liquid sprays are conveniently delivered from pressurized packs.

For administration by inhalation, the compounds of formula (I) or formula (I′) are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds of formula (I) or formula (I′) may take the form f a dry powder composition, for example, a powder mix of the, compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient may be employed.

The pharmaceutical compositions for use according to the invention may also contain other active ingredients such as antimicrobial agents or preservatives.

The compounds of formula (I) of formula (I′) may also be used in combination with other therapeutic agents, for example, other anti-infective agents. In particular, the compounds of formula (I) or formula (I′) may be employed together with well known antiviral agents, e.g., adenine arabinoside or interferon α.

The invention thus provides, in a further aspect, a combination comprising a compound of formula (I) or formula (I′) or a physiologically acceptable derivative thereof together with another therapeutically active agent, in particular, an anti-HBV agent.

The combinations referred to above may conveniently be presented for use in the form of a pharmaceutical formulation and thus the use of pharmaceutical formulations comprising a combination as defined above together with a pharmaceutically acceptable carrier therefor comprise a further aspect f the invention.

The individual components of such combinations may be administered either sequentially or simultaneously in separate or combined pharmaceutical formulations.

When the compound of the formula (I) or formula (I′) or a pharmaceutically acceptable derivative thereof is used in combination with a second therapeutic agent active against the same virus, the dose of each compound may be either the same or different from that when the compound of formula (I) or formula (I′) is used alone. Appropriate doses will be readily appreciated by those skilled in the art.

The compounds of formula (I) or formula (I′) and their pharmaceutically acceptable derivations may be prepared by any method known in the art for the preparation of compounds of analogous structure.

Four processes ((A) to (D)) for producing compounds of formula (I) are set forth as follows:

In one such process (A) a 1,3-oxathiolane of formula 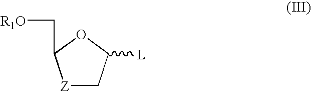
wherein R₁ is hydrogen or a hydroxyl protecting group as defined herein and the anomeric group L is a displaceable atom or group and is reacted with an appropriate base. Suitable groups L include alkoxy carbonyl groups such as ethoxy carbonyl or halogens, for example, iodine, bromine or chlorine or —OR′ where R′ is a substituted or unsubstituted, saturated or unsaturated alkyl group, e.g., a C₁₋₄-alkyl group such as methyl, r R′ is a substituted or unsubstituted aliphatic or aromatic acyl group, e.g., a C₁₋₆-aliphatic acyl group such as acetyl and an aromatic acyl group such as benzoyl.

The compound of formula (III) is conveniently reacted with the appropriate purine or pyrimidine base R₂—H (previously silylated with a silyating agent such as hexamethyldisilazine) in a compatible solvent such as methylene chloride using a Lewis acid (such as titanium tetrachloride or stannic chloride) or trimethyl-silytriflate.

The 1,3-oxathiolanes of formula (III) may be prepared, for example, by reaction of an aldehyde of formula (IV) with a mercaptoacetal of formula (V) in a compatible organic solvent, such as toluene, in the presence of an acid catalyst as a para-toluene sulfonic acid or a Lewis acid, e.g., zinc chloride.
HSCH₂CH(OC₂H₅)₂ (IV)
C₆H₅COOCH₂CHO (V)

The mercaptoacetals of formula (IV) may be prepared by methods known in the art, for example, G. Hesse and I. Jorder, “Mercaptoacetaldehyde and dioxy-1,4-dithiane,” Chem. Ber. 85 pp. 924-932 (1952).

The aldehydes of formula (V) may be prepared by methods known in the art, for example, E. G. Halloquist and H. Hibbert, “Studies on Reactions Relating to Carbohydrates and Polysaccharides, Part XLIV: Synthesis of Isomeric Bicyclic Acetal Ethers,” Can. J. Research 8, pp. 129-136 (1933).

In a second process (B) one compound of formula (I) is converted to another compound of formula (I) by base interconversion. Such interconversion may be effected either by simple chemical transformation (e.g., the conversion of uracil base to cytosine) or by an enzymatic conversion using, for example, a deoxyribosyl transferase. Such methods and conditions for base interconversions are well known in the art of nucleoside chemistry.

In a third process (C) the compounds of formula (I) may be prepared by the reaction of a compound of formula (VI) 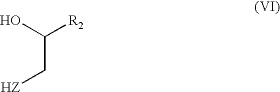
with a compound of formula (VII) 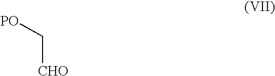
where P is a protecting group, followed by removal of the protecting group.

The compounds of formula (VI) may be prepared for reaction by a suitable epoxide (IX) 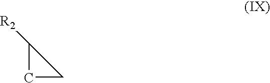
with an appropriate sulphur-containing compound, e.g., sodium thioacetate. Compounds of formula (IX) are either known in the art or may be obtained by analogous processes.

In a fourth process (D) a compound of formula (X) 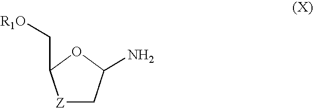
may be converted to a compound of formula (I) by conversion of the anomeric NH₂ group to the required base by methods well known in the art of nucleoside chemistry.

Many of the reactions described hereinabove have been extensively reported in the context of purine nucleoside synthesis, for example, in “Nucleoside Analogues—Chemistry, Biology and Medical Applications,” R. T. Walker et al., Eds. Plenum Press, New York (1979) at pages 193-223, the text of which is incorporated by reference herein.

It will be appreciated that the above reactions may require the use of, or conveniently may be applied to, starting materials having protected functional groups, and deprotection might thus be required as an intermediate or final step to yield the desired compound. Protection and deprotection of functional groups may be effected using conventional means. Thus, for example, amino groups may be protected by a group selected from arakyl (e.g., benzyl), acyl or aryl (e.g., 2,4-dinitrophenyl); subsequent removal of the protecting group being effected when desired by hydrolysis or hydrogenolsis as appropriate using standard conditions. Hydroxyl groups may be protected using any conventional hydroxyl protecting group, for example, as described in “Protective Groups in Organic Chemistry,” Ed. J. F. W. McOmie (Plenum Press, 1973) or “Protective Groups in organic Synthesis” by Theodora W. Greene (John Wiley and Sons, 1981). Examples of suitable hydroxyl protecting groups include groups selected from alkyl (e.g., methyl, t-butyl or methoxymethyl), aralkyl (e.g., benzyl, diphenylmethyl or triphenylmethyl), heterocyclic groups such as tetrahydropyranyl, acyl, (e.g., acetyl or penzoyl) and silyl groups such as trialkylsilyl (e.g., t-butyldimethylsilyl). The hydroxyl protecting groups may be removed by conventional techniques. Thus, for example, alkyl, silyl, acyl and heterocyclic groups may be removed by solvolysis, e.g., by hydrolysis under acidic or basic conditions. Aralkyl groups such as benzyl may be cleaved, for example, by treatment with BF₃ etherate and acetic anhydride followed by removal of acetate groups so formed at an appropriate stage in the synthesis. Silyl groups may also conveniently be removed using a source of fluoride ions such as tetra-n-butylammonium fluoride.

In the above processes, the compounds of formula (I) are generally obtained as a mixture of the cis and trans isomers.

These isomers may be separated, for example, by acetylation, e.g., with acetic anhydride followed by separation by physical means, e.g., chromatography on silica gel and deacetylation, e.g., with methanolic ammonia or by fractional crystillization.

With respect to the synthesis of the compounds of formula (I′), reference is made to Scheme 2 hereinafter which describes a synthesis for producing L-isomers of a dioxolane nucleoside analog.

1,6-Anhydro-L-gulose was prepared in one step from L-gulose by the treatment of L-gulose with an acid, e.g., 0.5N HCl in 60% yield (Evans, M. E., Earish, F. W., Carbohydr. Res. (1973), 28, 359). Without selective protection as was done before (Jeong, L. S.; Alves, A. J.; Carrigan, S. W.; Kim, H. O.; Beach, J. W.; Chu, C. K. Tetrahedron Lett. (1992), 33, 595 and Beach, J. W.; Jeong, L. S.; Alves, A. J.; Pohl, D; Kim, H. o.; Chang, C.-N.; Doong, S.-Li.; Schinazi, R. F.; Cheng, Y.-C.; Chu, C. K. J. Org. Chem. 1992, in press) (2) was directly converted to dioxolane triol (3) by oxidation with NaIO₄, followed by reduction with NaBH₄, which without isolation, was converted to isopropylidene derivative (4). Benzoylation to (5), deprotection to (6), and oxidation of diol (6) gave the acid (7). Oxidative decarboxylation of (7) with Pb(OAc)₄ in dry THF gave the acetate (8), the key intermediate in good yield. The acetate was condensed with the desired pyrimidines (e.g., silylated thymine and N-acetylcytosine) in the presence of TMSOTf to afford an α,β-mixture, which was separated on a silica gel column to obtain the individual isomers (9-12). Debenzoylation with methanolic ammonia gave the desired cytosine derivative 14 and the thymine derivative 13.

All key intermediates in Scheme 2 gave correct elemental analyses (±0.4%).

In summary, the asymmetric synthesis of (−)-L-β-dioxolane-C was accomplished via 8 steps from a chiral template 2.

Pharmaceutically acceptable salts of the compounds of the invention may be prepared as described in U.S. Pat. No. 4,383,114, the disclosure of which is incorporated by reference herein. Thus, for example, when it is desired to prepare an acid addition salt of a compound of the formula (I), the product of any of the above procedures may be converted in a salt by treatment of the resulting free base with a suitable acid using conventional methods. Pharmaceutically acceptable acid addition salts may be prepared by reacting the free base with an appropriate acid optionally in the presence of a suitable solvent such as an ester (e.g., ethyl acetate) or an alcohol (e.g., methanol, ethanol or isopropanol). Inorganic basis salts may be prepared by reacting the free base with a suitable base such as an alkoxide (e.g., sodium methoxide) optionally in the presence of a solvent such as an alcohol (e.g., methanol). Pharmaceutically acceptable salts may also be prepared from other salts, including other pharmaceutically acceptable salts of the compounds of the formula (I) using conventional methods.

A compound of formula (I) may be converted into a pharmaceutically acceptable phosphate or other ester by reaction with a phosphorylating agent, such as PoCl₃ or a suitable esterifying agent, such as an acid halide or anhydride, as appropriate. An ester or salt of a compound of formula (I) may be converted to the parent compound, for example, by hydrolysis.

Where the compound of formula (I) is desired as a single isomer, it may be obtained either by resolution of the final product or by stereospecific synthesis from isomerically pure starting material or any convenient intermediate.

Resolution of the final product or an intermediate or starting material therefore may be effected by any suitable method known in the art: see, for example, Stereochemistry of Carbon Compounds, by E. L. Eliel (McGraw Hill, 1962) and Tables of Resolving Agents, by S. H. Wilen.

The present invention is also directed to a novel methodology to prepare (−)3′-thia-2′,3′-dideoxycytidine or (−)5-fluoro-3′-thia-2′,3′-dideoxycytidine from (±)-3′-thia-2′,3′-dideoxycytidine or (±)5-fluoro-3′-thia-2′,3′-dideoxycytidine, respectively. The method takes advantage of the stereospecificity of the action of deoxycytidine deaminase (from either mammalian or bacteria sources) which converts deoxycytidine to deoxyuridine, and the separation of 3′-thia-2′,3′-dideoxycytidine and SddU. Preferably, the deamination is carried out at 37° C. for 16 hours. (−)3′-Thia-2′,3′-dideoxycytidine, (+)3′-thia-2′,3′-dideoxycytidine and (±)3′-thia-2′,3′-dideoxycytidine were examined for their HBV effect. The ID₅₀ of (+)3′-thia-2′,3′-dideoxycytidine, (±)3′-thia-2′,3′-dideoxycytidine and (−)3′-thia-2′,3′-dideoxycytidine against HBV were found to be approximately>0.5 μm, 0.1 μm and 0.02 μm respectively, which indicates that (−)3′-thia-2′,3′-dideoxycytidine is the primary form responsible for the anti HBV effect.

Cytotoxicity studies using CEM cells and dialyzed serum showed that (+)3′-thia-2′,3′-dideoxycytidine is more toxic than (±)3′-thia-2′,3′-dideoxycytidine, indicating that the cytotoxicity of (±)3′-thia-2′,3′-dideoxycytidine observed previously is primary due to the (+)3′-thia-2′,3′-dideoxycytidine, thus the therapeutic index of (−)3′-thia-2′,3′-dideoxycytidine against HBV should be much better than that of (±)3′-thia-2′,3′-dideoxycytidine or (+)3′-thia-2′,3′-dideoxycytidine.

It was unexpected that the (−) forms of 3′-thia-2′,3′-dideoxycytidine and 5-fluoro-3′-thia-2′,3′-dideoxycytidine would be more active than the respective (+) forms, since the naturally existing sugar moieties have the (+) configuration.

Without wishing to be bound by any particular theory of operability, it is possible that virus DNA polymerase is able to interact with the unnatural (−)-configuration. It can be expected that the therapeutic index of (−)3′-thia-2′,3′-dideoxycytidine should be better than the (+)- or (−)-form of 3′-thia-2′,3′-dideoxycytidine or its analogues.

The invention will be further described by the following examples which are not intended to limit the invention in any way.

### EXAMPLES

### Example 1

### Cis-and trans-2-hydroxymethyl-5-(cytosin-1-yl)-1,3-oxathiolanes

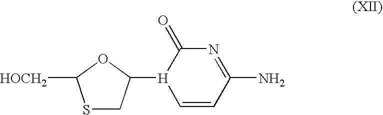

(a) Trans-: 375 mg of the trans-(XI) 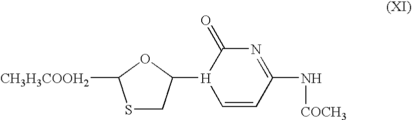
was dissolved in 100 ml of methanolic ammonia at 24° C. and after stirring for 16 hours, the solvent was removed in vacuo and the residue crystallized with ether. It was recrystallized from ethanol-ether to yield 174 mg of pure product, m.p.>220° (dec). It was characterized by ¹H and ¹³C NM.
¹H NMR δ(ppm in DMSO-d₆);
7.57 (d. 1H; C₆—H)
7.18 (d. 2H; C₄—NH₂)
6.30 (dd. 1H; C₅—H)
5.68 (d. 1H; C₅—H)
5.48 (t, 1H; C₅—H)
5.18 (t, 1H; C₂—CH₂—OH)
3.45 (m. 3H; C₂—CH2OH+C₄H)
3.06 (dd. 1H; C₄—H)
U.V.: (CH₃OH) λ max: 270 mm

³C NMR (DMSO-d₆, Varian.: XL-300); δ in ppm:

| C₂ | C₄ | C₅ | C₆ | C₅ | C₄ | C₂ | CH₂OH |
|---|---|---|---|---|---|---|---|
| 154.71 | 165.7O | 93.47 | 140.95 | 87.77 | 36.14 | 86.80 | 64.71 |

(b) Cis-: treating 375 mg. of cis-(XII) by the same preceding procedure led to 165 mg of pure product after recrystallization from ethanol-ether, m.p. 171-173° It was characterized by ¹H and ³C NMR.
¹H NMR: δ(ppm in DMSO-d₆):
7.80 (d, 1H; C₆—H)
7.20 (d,2H; C₄—NH₂)
6.18 (t, 1H; C₅—H)
5.70 (d, 1H; C₅—H)
5.14 (t, 1H; C₂—CH₂OH)
3.71 (m, 2H; C₂—CH₂OH)
3.40 (dd, 1H; C₄—H)
2.99 (dd, 1H; C₄—H).
U.V.: (CH₃OH) δ max: 270 nm

### Example 2

### Preparation of Bis-Pivaloyl Protected Cis-2-Buten-1,4-Diol

A 10 L, 3-neck flask was flame dried under vacuum and filled with argon. A positive flow of argon was maintained while the flask was fitted with a dried mechanical stirrer and charged with 100 g (0.15 eq) of DMAP, 3200 ml (39.7 moles, 7 eq) of anhydrous pyridine, and 500 g (5.67 moles, 1.00 eq) of cis-2-butene-1,4-diol. The flask was fitted with a septum. The contents were stirred at 0° C. under continuous flow of argon.

1500 ml (2.15 eq) of pivaloyl chloride was added slowly, via a cannula, (in 100-200 ml portions) maintaining a low temperature and limiting gas evolution. The reaction mixture was allowed to stir for an additional hour at 0° C. under argon, and was subsequently quenched by the addition of crushed ice.

The solution was decanted from the solids and the pyridine was evaporated in vacuo. The remaining material was dissolved in ether and washed once with saturated CuSO₄ solution, twice with saturated NaHCO₃ solution and twice with water. The ethereal solution was dried over MgSO₄, filtered, evaporated, and placed on a vacuum pump overnight.

The solid residue was dissolved in water and the resulting solution was extracted twice with ether. The ether solution was washed, dried over MgSO₄, filtered, evaporated, and further dried in vacuo.
Product mol. wt.=256.341 (C₁₄H₂₄O₄)
Theoretical yield=1,453 g
Actual yield=1,439 g
% yield=99.0%

### Example 3

### Coupling of Pivalate Acetate with Bis(silylated)-5-Fluoro-Cytosine

An appropriate size dry flask was charged with 199 ml methylene chloride (dried in a still) and 6.61 g (corresponding to 0.0224 mols pure acetate, 1 eq) of an 88.841% (by GC analysis) of acetate under nitrogen. A second dry appropriate size flask was charged with 24 ml methylene chloride (dried in a still) and 7.67 g (0.0278 mols, 1.25 eq) of bis-silylated-5-fluorocytosine under nitrogen. After cooling the fluorocytosine mixture to 020 C., a 1.0M solution of Lewis acid in methylene chloride was added via a syringe. This mixture was then brought up to room temperature and subsequently cannulated into the acetate mixture over a 20 minute period. The reaction was monitored by thin layer chromatography using an anisaldehyde stain (anisaldehyde stain: 5% anisaldehyde, 5% concentrated H₂SO₄, 90% absolute ethanol) for visualization; disappearance of starting materials using hexane: ether, 4:1 indicated completion (45 minutes). The yellow mixture was quenched by diluting with 250 ml methylene chloride (1.5 times the total reaction solvent volume) then 47 ml concentrated ammonium hydroxide was added slowly with the addition of ice pieces to control temperature. The solution separated into two layers, the top aqueous layer which contains some solid and the bottom organic layer which is a yellow solution. The organic layer was divided into three portions. Each portion was filtered through 1 inch of silica gel using a 300 ml fritted funnel (fresh silica gel for each portion). The silica gel layers were then washed with 6:1 ethyl acetate: absolute ethanol. The organic layers were combined and the solvents were evaporated by rotary evaporation. The product was precipitated out by adding ether and a drop or two of methylene chloride.

Filtration of the product and subsequent drying in vacuo gave a 51% pure product yield (100% β). Repeating the precipitation process allowed for the isolation of a second crop of material, bringing the total yield to 59%. The solid is a white powder.

The reaction can be performed using two different methods:
(1) Adding a Lewis acid to an acetate/silylated base mixture.
(2) Pre-mixing a Lewis acid and silylated base and adding to this mixture the acetate.

In order to obtain reasonable results, the acetate must be at least 80% pure by GC. An amount of 92.1 g of an oil containing 85.3% of acetate by GC, and 64.62 g of bis(trimethylsilyl)-5-fluorocytosine were dissolved in 1270 ml of dry CH₂Cl₂, all under an argon atmosphere. Next, 430 ml of a 1M Lewis acid/CH₂Cl₂ solution was added in 60 ml portions over 25 minutes. The resulting mixture was stirred for an additional 90 minutes, during which time the color changed from yellow to orange, and finally turning brown. This mixture was then diluted with 2000 ml of CH₂Cl₂ and quenched slowly with 400 ml of concentrated NH₄OH (small amounts of ice were added to regulate the exothermicity) Upon quenching, a precipitate formed, gradually the cloudy solution separated into two layers; an aqueous layer containing some suspended solid (upper) and an organic layer (lower).

The quenched mixture was then filtered through 1-2 inches of silica gel (using a 3000 ml fritted funnel). During this process, the silica was changed 3 times due to hydration. All silica gel layers were then washed 4 times with 700 ml of a 6:1 EtoAc/EtOH mixture. The solvent was then evaporated from both the CH₂Cl₂ and EtOAc layers in vacuo.

The solid residue from the CH₂Cl₂ layer was partially dissolved with Et₂O, filtered, and the undissolved solid was washed with additional ether to give 44.46 g (49%) of a white solid. The same procedure for the residue from the EtOAc layer resulted in 21.76 g (24%) of an off-white powder.

### Example 4

### Monitored Preparation of 2-t-Butyldiphenyl-silyloxymethyl-5-Oxo-1,3-Oxathiolane

Silylated glycolaldehyde (141.41 g, 0.474 mol) is dissolved in toluene (220.0 ml) in a three neck, 3000 ml round bottom flask. The flask was equipped with a stir bar, glass stopper, rubber septa, and a Dean-Stark trap to remove water during the reflux. Thioglycolic acid (33.93 ml, 0.488 mol) was added to the solution, and then heat was applied. The reaction usually takes approximately two hours to go to completion, and can be monitored by TLC (3:1 hexane:ether). By TLC the aldehyde (Rf approximately 0.3) appears just below the lactone product using a UV lamp for visualization, and when its trailing “tail” disappears, the reflux can be stopped. GC analysis (30 m SPB-5 on fused silica cap. col) program 80° C./5 minutes—10° C./minutes—310° C. is performed. The aldehyde peak appears at approximately 22.13 minutes, the lactone at approximately 27.94 minutes. This can be used to determine the final amount of aldehyde left in the reaction mixture.

When the amount of aldehyde left is negligible, cool the solution to room temperature and transfer to a separatory funnel. Wash two times with water, then extract the water layers with ether to remove residual product. Wash the combined organic layers with water once more, then dry over MgSO₄, filter, and concentrate. A colorless oil results, which will almost all solidify under vacuum (usually left on pump for two days).

### Example 5

### Standardized Procedure for the Desilylation of 1″-O-(tert-Butyldiphenylsilyl)-3′-thia-5-fluorocytidine to 5-fluoro-3′-thia-2′,3-dideoxycytidine

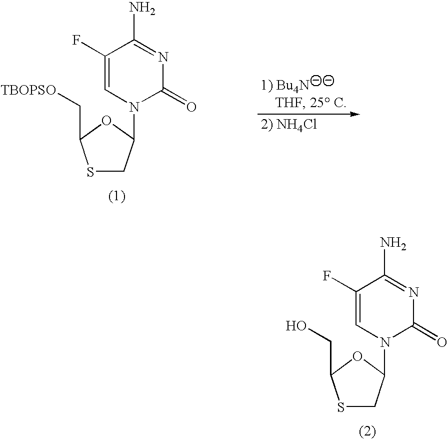

Procedure: 1″-O-(tert-Butyldiphenylsilyl)-3′-thia-5-fluorocytidine (1) 44.46 g; 91.54 mol) was dissolved in 250 mL of dry THF. Tetrabutylammonium fluoride (105 mL; 105 mmol) was added dropwise over a period of 3-5 minutes as a 1.0 M solution in THF. The progress of the reaction was monitored by TLC using 6:1 EtOAc-EtOH; visualization was accomplished by UV as well as staining with PMA following by charring. TLC analysis of the reaction mixture showed four spots: at a baseline; at R_{f}0.3 (corresponding to (1)); oblong-shaped spot); at R_{f}0.75 (corresponding to (2)); at the solvent front (presumably TBDPS—F). The reaction mixture was stirred at room temperature; all the starting material was consumed after 45 minutes. The mixture was treated with 20 mL of saturated NH₄Cl followed by stirring for 1 hour. It was then filtered through a bed of silica gel (3 inch depth in a 350 mL fritted funnel); the silica gel was washed with 600 mL of 2:1 EtOAc-EtOH. The filtrate was reduced to a volume of approximately 100 mL by rotary evaporation during which precipitation of a white solid was observed. Filtration of the mixture gave a white powder which was a mixture of the desired product contaminated with ammonium salts. The crude solid was recrystallized from EtOH—CH₂Cl₂ to give 18.15 g (80% yield) of product. A second crop (2.09 g; 9% yield) of crystals was obtained, giving a total of 20.24 g (89% total yield) of 3′-thia-5-fluorocytidine (2): ¹H-NMR (DMSO-d₆; 300 MHz) 8.20 (d, J=7.3 Hz, 1H), 7.82 (br s, 1H), 7.58 (br s, 1H), 6.15-6.12 (m, 1H), 5.41 (t, J=5.7 Hz, 1H), 5.18 (m, 1H), 3.81-3.69 (m, 2H), 3.44 (AB dd, J=5.4 Hz, J=5.4 Hz, J=11.8 Hz, 1H), 3.12 (AB dd, J=4.3 Hz, J=4.3 Hz, J=11.8 Hz, 1H); α-isomer: ¹H-NMR (DMSO-d₆; 300 MHz) 7.83 (br d, J=7.0 Hz, 2H), 7.56 (br s, 1H), 6.29-6.27 (m, 1H), 5.59 (t, J=5.1 Hz, 1H), 5.19-5.16 (m, 1K), 3.57-3.38 (m, 2H), 3.17-3.12 (m, 2H).

The ratio of α:β-isomers present may be more accurately determined by HPLC analysis of the product on a Rainin Dynamax Phenyl column using a 97:3H₂O—CH₃CN isocratic solvent system. The analysis was performed on a Shimazu LC-6A liquid chromatography system fitted with a Rainin Dynamax Phenyl column (column length: 25 cm; internal diameter: 4.6 mm; particle size 8 μm; pore size: 6 Å) and linked to a UV-vis spectrophotometric detector (λ_{obs}=265 nm). Elution was accomplished using an isocratic solvent system of 97.3 water-CH₃CN at a flow rate of 0.8 mL/minute. Under these conditions the β-isomer elutes first with a retention time (R_{T})=45.5 minutes while the α-isomer has Rₜ=54.00 minutes.

### Example 6

### Diprotection of 1,4-Butenediol with t-Butyl Diphenylsilyl Chloride

A dry appropriate size flask was charged with 8.20 ml diol (0.996 mols, 1 eq), 2.44 g DMAP (0.020 mols, 0.2 eq), and 500 ml methylene chloride (0.2 molar solution; can be done at elevated concentrations up to 0.5 molar). This solution was reduced in temperature to 0° C. then 41.8 ml triethyl amine (0.300 mmols, 3 eq) was added via addition funnel. The mixture needed to be shaken by hand occasionally due to viscosity and was stirred for three hours (monitoring by thin layer chromatography (4:1 hexane; ether; UV lamp for visualization) for completion. The reaction mixture was concentrated by rotary evaporation. The residue was taken up in ether and washed twice with 10% hydrochloric acid (HCl), twice with saturated aqueous sodium bicarbonate solution, and once with brine. The organic layer was dried over anhydrous magnesium sulfate, filtered and concentrated via rotary evaporation to yield 57.26 g of diprotected product.

2′,3′-Dideoxy-5-fluoro-3′-thiauridine: ¹H NMR (DMSO-d⁶) 11.89 (1H, broad, NH), 8.33 (1H, d, H₆, J=7.5 Hz), 6.15 (1H, t, H, J=3.9 Hz), 5.44 (1 H, t, OH, J=5.7 Hz) 5.19 (1H, t, H₄, J=3.6 Hz), 3.75 (2H, m, 2H₅), 3.43 (1H, dd, 1H₂, J=5.7 and 12.0 Hz), 3.25 (1H, dd, 1H, J=4.2 & 12.0 Hz); mp 158-159° C.; Anal. Calc. for C₈H₉O₄N₂SF:C₁38.71; H, 3.65; N, 11.29; S, 12.92.

Found: C, 38.79: H, 3.68; N, 11.23; S, 12.82.

2′,3′-Dideoxy-5-fluoro-3′-thiacytidine: ¹H NMR (DMSO d⁶) 8.18 (1H, d, H₆, J=8.4 Hz) 7.81 and 7.57 (2H, broad NH2), 6.12 (1H, dd, H₁, J=5.7 and 4.2 Hz), 5.40 (1H, t, OH, J=5.7 Hz), 5.17 (1H, t, H₄, J=3.6 Hz), 3.74 (2H, M, 2H₅), 3.41 (1H, dd, 1 H2, J=5.7 and 11.7 Hz)., 3.11 (1H, dd, 1 Hz₂, J=4.2 and 11.7 Hz); mp 19.5-196° C.; Anal. Calc. for C₈H₁₀O₃N₃SF:C, 38.86; H, 4.08; N, 17.00; S, 12.97. Found: C, 38.97; H, 4.07; N, 16.93; S, 12.89.

### Example 7

### In Vitro Assay for Antiviral Activity

2.2.15 cells were inoculated at a density of 3×10⁵ cells/5 ml in 25 cm² flask. Drugs were added to the medium 3 days after the inoculation. Cells were grown in the presence of drugs for 12 days With changes of medium every 3 other days. At end of the incubation, the medium was centrifuged (10 minutes, 2,000×g) and the supernatant was subjected to a final concentration of 10% (wt/vol) PEG 8,000. The virus was pelleted (10 minutes 10,000×g). The pellet was resuspended at 1/100th the original volume in TNE buffer (10 mM Tris pH 7.5, 100 EM NaCl, 1 mM EDTA). The suspension was adjusted to 1% SDS and 0.5 mg/ml proteinase K and incubated for 2 hours at 55° C. The digest was extracted with phenol, chloroform and the DNA was precipitated with ethanol. The DNA pellet was dissolved in TE₈₀ (10 mM Tris pH 8.0, 1 mM EDTA) and electrophoresed in a 0.8% agarose gel, followed by blotting onto Hybond-N membrane (Amersham). The filter was hybridized with ³²P-labeled HBV DNA probe, washed with 2×SSC containing 0.2 t SDS at room temperature for 1 hour, 0.1×SSC containing 0.2% SDS at 55° C. for ½ hour and autoradiographed.

### Example 8

### Isolation and Characterization of DNA

Drug-treated cells were lysed with a buffer containing 10 mM Tris pH 7.5, 5 mM EDTA, 150 mM NaCl and 1% SDS. The cell lysate was digested with 100 μg/ml proteinase K at 55° C. for at least 2 hours and deproteinized by extraction with phenol. Nucleic acids were precipitated with 2 volumes of ethanol. The pellet of nucleic acid was redissolved in 10 mM Tris pH 8.0, 1 mM EDTA followed by 100 μg per ml RNase treatment at 37° C. for 1 hour. Concentrated ammonium acetate was added to the aqueous phase to yield a final 0.4 M ammonium acetate solution. The nucleic acids were precipitated with ethanol.

### Example 9

### Isolation of RNA

Total cellular RNA was isolated according to Chomczyndki et al. (Chomczynsk, P., and Sacchi, N., “Single Step Method for RNA Isolation by Acid Guanidinium Thiocyanate-Phenol-Chloroform Extraction,” Analytical Biochemistry, 162: 156-159, 1987). The RNA (20 μg per lane) was electrophoresed through 1% agarose gel containing 1.1×formaldehyde and transferred to Hybond-N membrane. The immobilized RNA was hybridized with ³²P-labeled HBV DNA probe and the membrane was autoradiographed as described above.

### Example 10

### Cytotoxicity

CEM (T lymphoblastoid cells) cells were inoculated in 5 ml RPMI 1640 supplemented with 5% fetal bovine serum at a concentration of 2×10³ cells per ml. The cells were incubated with different concentrations of compounds for 4 days. At day 4, the cell number was determined by a coulter counter or a hemocytometer.

### Example 11

### Determination of mtDNA Content by Quick Blot Procedure

The CEM cells (5×10⁴) cells were collected and freeze-thawed three times. The cell lysate was incubated with RNase (10 μg/ml) at 37° C. for 30 minutes. The sample was treated with proteinase K (100 μg/ml) at 55° C. for 1 hour. A 0.8 volume of supersaturated Nal (2.5 gNaI in 1 ml hot water) was added to the sample and heated at 90° C. for 10 minutes. The DNA was immobilized on nitrocellulose paper by using a slot blot apparatus (Schleicher & Schuell, Keene, NH). The mtDNA on the nitrocellulose paper was detected with a mtDNA specific probe as described previously (Chen, C. H., Cheng, Y. C., “relayed Cytotoxicity and Selective Loss of Mitochondria DNA in Cells Treated with the Anti-Human Immunodeficiency Virus Compound 2′,3′-Dideoxycytidine,” J. Biol. Chem. 264: 11934-11937, 1989).

### Example 12

### Comparative Potency of Deoxycytidine Analogs as Inhibitors of HBV In Vitro

2.2.15 cell lines was used to evaluate the antiviral activities of modified dideoxycytidine analogs—ddC (obtained from Pharmacia, Inc.), D4C, 3′-fluoro-2′,3′-dideoxycytidine (3′-FddC), 5-fluoro-3′-thia-2′,3-dideoxycytidine (5-FSddC) and 3′-thia-2′,3′-dideoxycytidine (SddC).

The formulas for ddC, d4c and 3′-FddC are as follows:

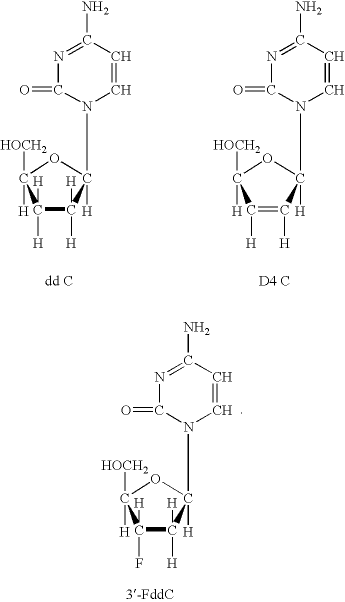

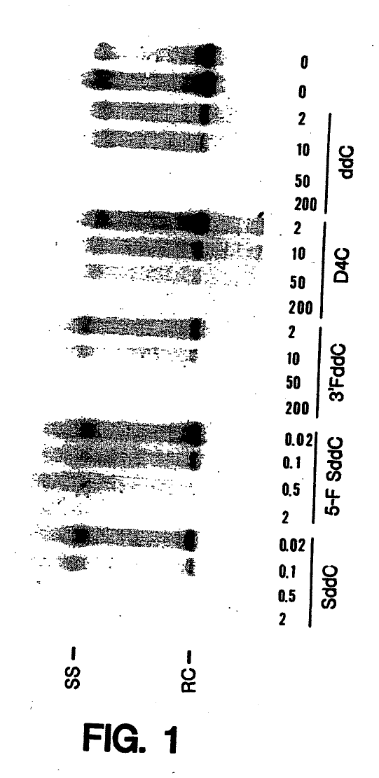

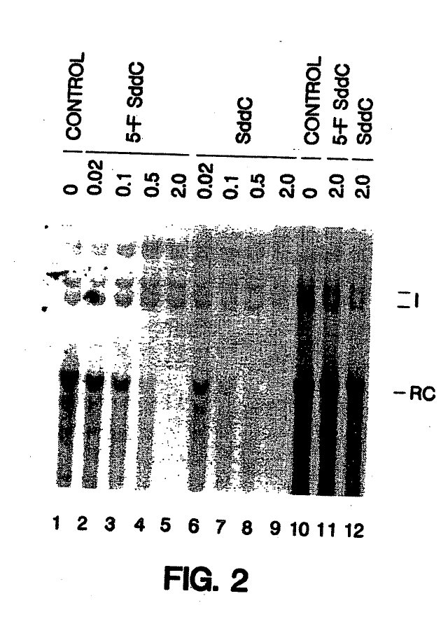

The antiviral effects were measured by analysis of extracellular HBV DNA (FIG. 1). The experiment revealed that the amount of extracellular UBV DNA decreased in a dose dependent manner. The inhibitory concentration for a 50% decrease in viral replication (HBID₅₀) of these compounds are presented in Table 1. At concentration of 2 μM, both 5-fluoro-3′-thia-2′,3-dideoxycytidine and 3′-thia 2′,3′-dideoxycytidine completely inhibited the replication of HBV, approximately 50% inhibition by 3′-FddC, whereas neither ddC nor D4C had any impact effect. Episomal HBV DNAs in 2.2.15 cells treated with different concentrations of 5-fluoro-3′-thia-2′,3′-dideoxycytidine and 3′-thia-2′,3′-dideoxycytidine were also traced. Cellular DNAs were digested with Hind III that does not cleave within HBV genome, and subjected to Southern analysis using P³²-labeled HBV DNA as a probe. The chromosomally integrated HBV DNA genome and the episomal DNA can be separated in the gel and can be differentially quantified. While episomal DNA decreased in a dose dependent manner as extracellular HBV DNAs, both the amount and the restriction enzyme pattern of the chromosomally integrated HBV genome were unaltered (FIG. 2).

### Example 13

### Comparative Cytotoxicity and Effect on Mitochondria DNA Content of Deoxycytidine Analogs

ED₅₀s (concentration of compounds which caused a 50% reduction in cell density) are shown in Table 1. Mitochondria DNA content was measured by slot blot hybridization analysis. MtID50s (concentration of compounds which caused a 50% reduction in mitochondria DNA content) are also shown in Table 1. Both 5-fluoro-3′-thia-2′,3-dideoxycytidine and 3′-thia 2′,3′-dideoxycytidine inhibited HBV replication at concentrations hundreds or thousands fold lower than concentrations which induced cytotoxicity in CEM cells. Moreover, at concentrations which caused cessation of HBV replication, no effect on mitochondria DNA synthesis was observed. This contrasts with ddC (a potent polymerase gamma inhibitor) which had a HBID₅₀ of 3.8 μM but a low MtID₅₀, at 0.022 μM. D4C had a lower value of MtID₅₀ (2.0 μM) than HBID₅₀ (2.4 ═M). Thus, D4C would be expected to have a mitochondria effect before the anti-HBV effect was achieved as the same phenomenon observed in treatment with ddC. 3′-FddC had a greater selective index than both ddC and D4C. However, the ED₅₀ versus the MTID₅₀ ratio is about the same as ddC, suggesting 3′-FddC is not the drug of choice either.

TABLE 1
Comparative Potencies of Dideoxycytidine
Analogues as Monitored by Anti-HBV,
Cytotoxicity and Mitochrondria DNA Effect

| | HBID₃₀¹ | MTID₃₀² | ED₃₀³ | | |
|---|---|---|---|---|---|
| Compounds | (μM) | (μM) | (μM) | S.I.⁴ | ED₃₀/MtID₃₀ |
| ddC | 3.6 | 0.022 | 10 | 2.6 | 454 |
| D4C | 2.4 | 2 | 22 | 9.2 | 11 |
| 3*−FddC | 1.6 | 0.11 | 45 | 28.1 | 409 |
| 3′thia-2′,3′- | 0.05 | 47 | 37 | 740 | 0.79 |
| dideoxycyti- | | | | | |
| dine | | | | | |
| 5-fluoro-3′- | 0.1 | >200 | >200 | >2000 | NA |
| thia-2′,3′- | | | | | |
| dideoxycyti- | | | | | |
| dine | | | | | |

^{1concentration which caused a 50% reduction in EEV replicatian}
^{2concentration which caused a 50% reductian in mitochondria DNA content}
^{3concentration which caused a 50% reduction in cell density}
^{4Selective Index (EO}₃₀/HBID₃₀)
^{*nonapplicable}

### Example 14

### Reversibility of 5-fluoro-3′-thia-2′,3-dideoxycytidine and 3′ thia-2′,3′-dideoxycytidine

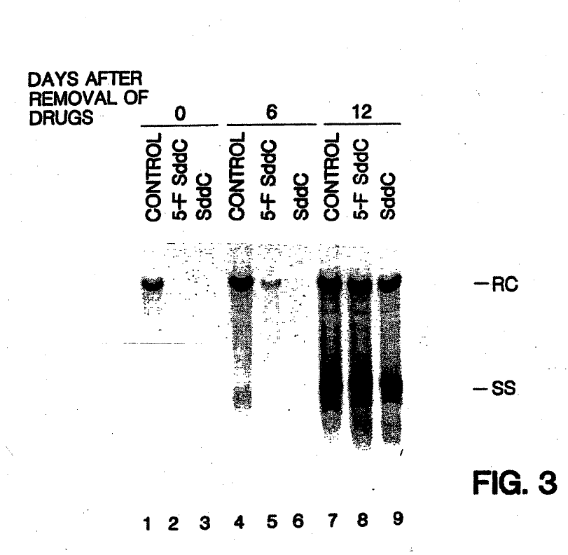

To determine whether the antiviral effect was reversible, 2.2.15 cells that were treated with a 2.0 μM concentration of 5-fluoro-3′-thia-2′,3-dideoxycytidine and 3′-thia-2′,3′-dideoxycytidine for 12 days were incubated for additional 6 or 12 days in the absence of the compound. After 6 days of drug free incubation, HBV DNA could again be identified extracellularly (FIG. 3), though not as much as those in control untreated cells. After 12 days of drug-free incubation, both extracellular (FIG. 3) and intracellular episomal HBV DNAs (FIG. 2) bounced back.

### Example 156

### Detection of HBV-Specific RNAs in Both Control and Drug Treated 2.2.15 Cells

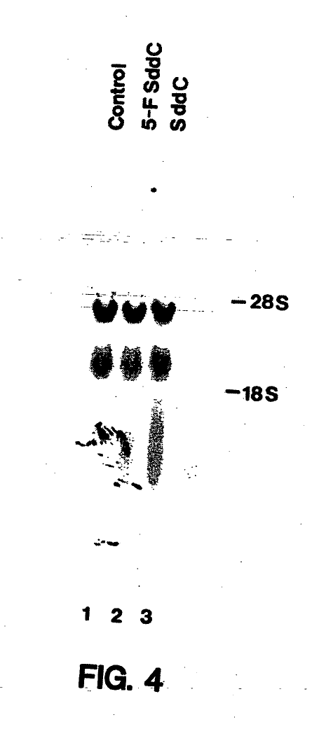

Northern blot analysis was performed to analyze HBV RNA transcripts. Three major transcripts of approximately 3.5, 2.5, and 2.1 Kb were detected in the total cellular RNA extract. The HBV specific transcripts were not affected at all by 2.0 μM 5-fluoro-2′-thia-2′,3′-dideoxycytidine and 3′-thia-2′,3′-dideoxycytidine treatment (FIG. 4).

### Example 16

### Comparative Potency of Pyrimidine 3′-thia 2′,3′-dideoxynucleoside Analogs as Inhibitors of HBV In Vitro

Various analogs (Table 2) of 3′-thia-2′,3′-dideoxycytidine were tested in 2.2.15 cells for anti-HBV activity. At 1.0 μM, none of the 3′-thia 2′,3′-dideoxyuridine analogs was active against HBV replication (FIG. 5). Among the 3′-thia 2′,3′-dideoxycytidine analogs tested, both 3′-thia-2′,3′-dideoxycitidine (R₅═H) and 5-fluoro-3′-thia-2′,3-dideoxycytidine (R₅═F) had the most potent inhibitory effect on HBV replication. At 1.0 μM, 5-Br, 5-Cl, and 5-CH₃ analogs are not active, whereas the 5-I analog reduced the HBV production by at least 2 fold.

TABLE 2

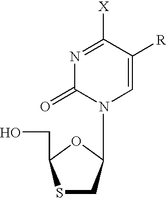

| | X | R |
|---|---|---|
| | OH | H |
| | OH | Me |
| | OH | F |
| | OH | Cl |
| | OH | Br |
| | OH | I |
| 3′-thia-2′,3′-dideoxy-cytidine | NH₂ | H |
| | NH₂ | Me |
| 5-Fluoro-3′-thia-2′,3′-dideoxy-cytidine | NH₂ | F |
| | NH₂ | Cl |
| | NH₂ | Br |
| | NH₂ | I |

### Example 17

### Preparation of (—)3′-thia-2′,3′-dideoxycytidine From (±)-3′-thia-2′,3′-dideoxycytidine

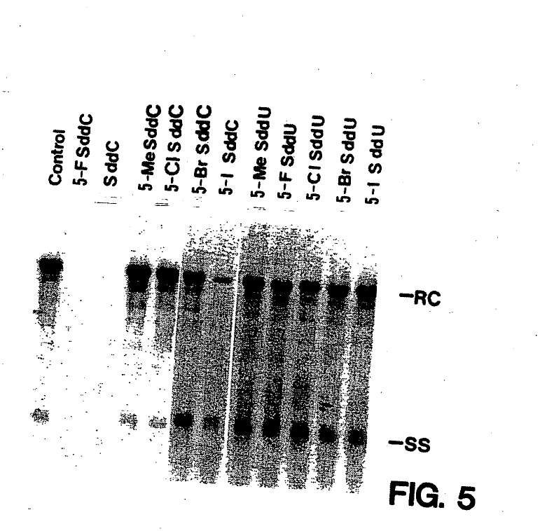

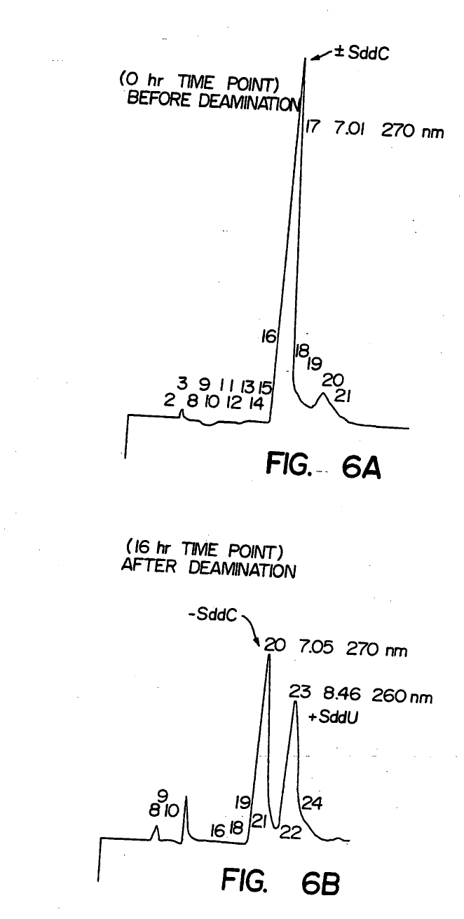

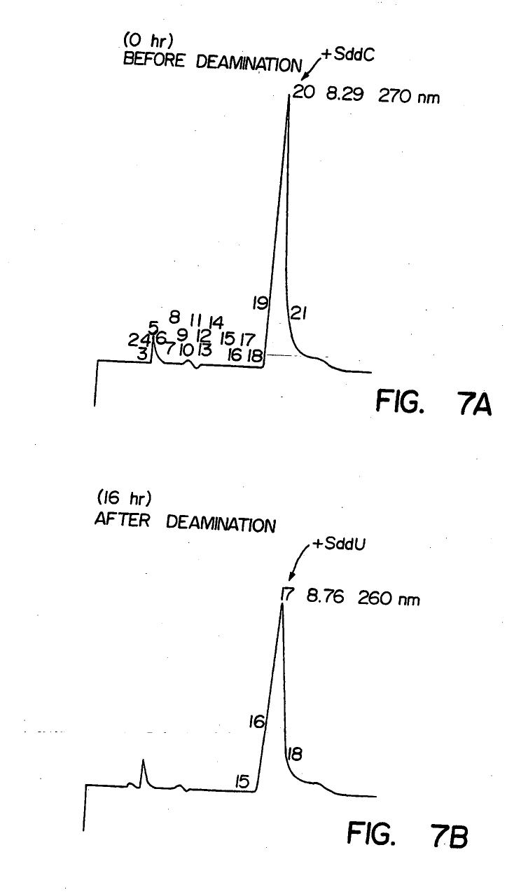

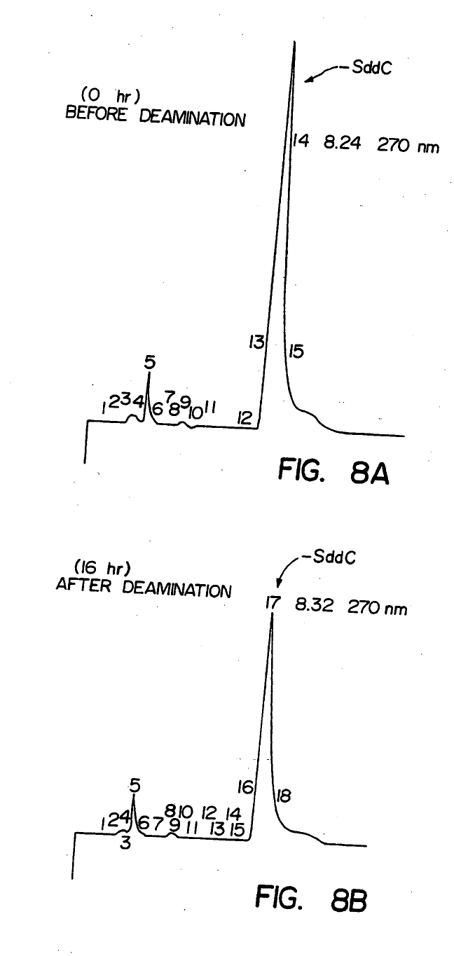

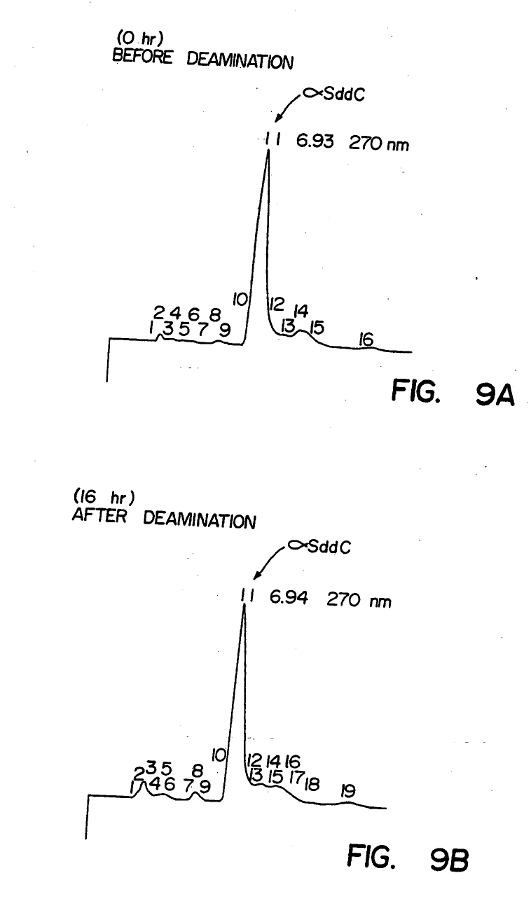

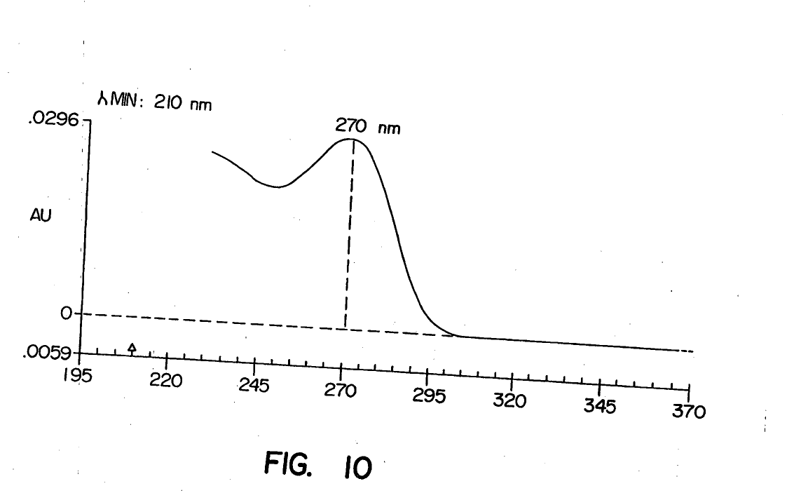

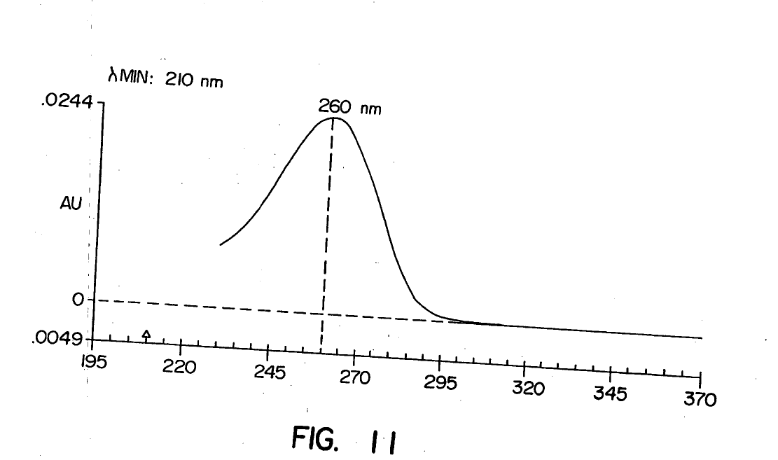

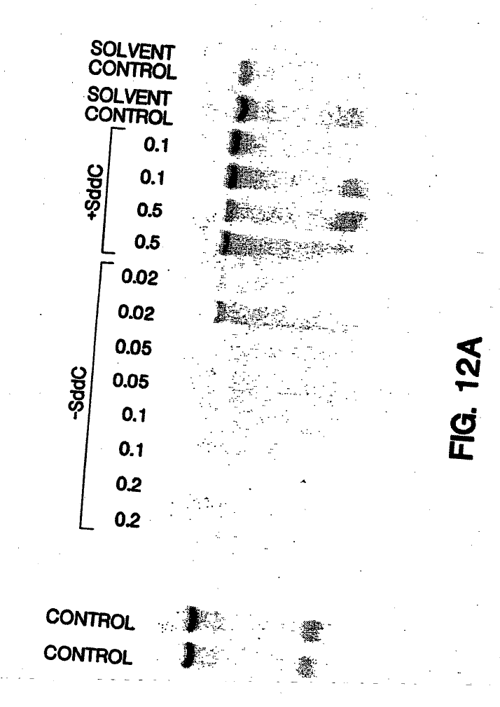

Since the (−)3′-thia-2′,3′-dideoxycytidine was not available by chemical synthesis and the (+)-3′-thia-2′,3′-dideoxycytidine and (−)3′-thia-2′,3′-dideoxycytidine cannot be separated by conventional methods, applicants developed a novel methodology to separate (−)3′-thia-2′,3′-dideoxycytidine from (+)3′-thia-2′,3′-dideoxycytidine by taking the advantage of the deoxycytidine (structure <1>) deaminase (from either bacteria or mammalian sources). (+)3′-Thia-2′,3′-dideoxycytidine can be selectively deaminated by dCdR (deoxycytidines deaminase), based on HPLC retention time and UV spectrum and leave (−)3′-thia-2′,3′-dideoxycytidine intact from (±)3′-thia-2′,3′-dideoxycytidine mixture. The reaction mixture which contains (+)-3′-thia-2′,3′-dideoxycytidine (retention time 8.46 minutes, UVₘₐₓ 260 nm) and (−)3′-thia-2′,3′-dideoxycytidine (retention time 7.05 minutes, UVₘₐₓ 270 nm) can then be separated by HPLC (FIG. 6A and FIG. 6B, using 100 mM NH₄Ac to achieve better separation). A control reaction which has only (+)3′-thia-2′,3′-dideoxycytidine proved that (+)3′-thia-2′,3′-dideoxycytidine is the substrate of CdR deaminase and can be almost 100% deaminated (FIG. 7A and FIG. 7B). (±)3′-Thia-2′,3′-dideoxycytidine can be deaminated only up to approximately 50%; no further deamination has been observed. After HPLC separation, (−)3′-Thia-2′,3′-dideoxycytidine was resubjected to deaminase and showed no sign of deamination at all (FIGS. 8A and 8B). α-3′-Thia-2′,3′-dideoxycytidine has a different retention time from (+)3′-thia-2′,3′-dideoxycytidine on HPLC (α-3′-thia-2′,3′-dideoxycytidine has a retention time of approximately 6.9 minutes compared with (+)3′-thia-2′,3′-dideoxycytidine which has a retention time of approximately 8.3 minutes using no salt solvent system under the same condition as the one used in the case of (+)3′-thia-2′,3′-dideoxycytidine and (−)3′-thia-2′,3′-dideoxycytidine). However, no deamination occurred (FIG. 9A and FIG. 7B) under the same condition that (+)3′-thia-2′,3′-dideoxycytidine was completely deaminated.

A similar pattern of deamination of (±)5-fluoro-3′-thia-2′,3′-dideoxycytidine was also observed and the product, (+)5-fluoro-3′-thia-2′,3′-dideoxyuridine and (−)5-fluoro-3′-thia-2′,3′-dideoxycytidine was confirmed using the chemically synthesized 5-fluoro-3′-thia-2′,3′-dideoxyuridine as a standard.

### Example 18

### Assay Condition (HPLC Analysis)

The reaction mixture contained 25 mM Tris-HCl, pH 8.0, 0.1 mM of 3′-thia-2′,3′-dideoxycytidine and 0.2 unit of human cytidine deaminase (unit definition=the amount of enzyme which converts 1 n mole of cytidine to uridine per minute at 37° C.) in a total volume of 50 μl. After each time point, 100 μl of acetonitrile were added to the reaction mixture and agitated. The precipitated proteins were removed by centrifugation, and the supernatent was lyophilized to dryness. The samples were reconstituted with 200 μl of HPLC mobile phase buffer and analyzed on an Alltech RP-18 column. 3′-Thia-2′,3′-dideoxycytidine and SddU were detected at a UV absorption wavelength of 270 nm. The mobile phase was 10% acetonitrile/water unless specified otherwise, and the flow rate was 0.8 ml/minutes.

### Example 19

### In Vitro Assay for Antiviral Activity

The 2.2.15 cells (clonal cells derived from Hep G2 cells that were transfected with a plasmid containing HBV DNA) that secrete hepatitis B virions were used. The 2.2.15 cells were. maintained in minimal essential medium (MEM) supplemented with 10% fetal bovine serum. Cells were incubated at 37° C. in a moist atmosphere containing 5% CO₂ in air. The 2.2.15 cells were inoculated at a density of 3×10⁵ cells/ 5 ml in 25 cm² flask. The compounds studied were added to the medium three days after inoculation. Cells were grown in the presence of drugs for 12 days with changes of medium every three days. After incubation, the medium was centrifuged (10 minutes, 2,000×g) and polyethylene glycol (Mᵣ 8,000) was added to the supernatant to a final concentration of 10% (wt/vol.) The virus was pelleted (10 minutes 10,000×g). The pellet was resuspended at 1/100th the original volume in TNE buffer (10 mM Tris pH 7.5, 100 mM NaCl, 1 mM EDTA). The suspension was adjusted to 1% SDS and 0.5 mg/ml proteinase K and incubated for 2 hours at 55° C. The digest was extracted with phenol-chloroform and the DNA was precipitated with ethanol. The DNA pellet was dissolved in TE₈₀ (10 mM Tris HCl pH 8.0, 1 mM EDTA) and then electrophorsed in a 0.8% agarose gel followed by blotting onto Hybond-N membrane (Amersham, Arlington, Ill.). The filter was hybridized with ³²P-labeled HBV DNA (Bam HI insert from plasmid Pam6 (American Type Culture Collection, Rockville, Md.) probe, washed with 2×SSC containing 0.2% SDS at room temperature for 1 hour, 0.1×SSC containing 0.2% SDS at 55° C. for ½ hour and then autoradiographed. The intensity of the autoradiographic bands was quantitated by a scanning densitometer. The amount of HBV-specific DNAs was similar in separate experiments performed in duplicate. HBID₅₀ was defined as the drug concentration that inhibited HBV viral DNA yield in the medium by 50%. The values were obtained by plotting percentage inhibition compared with control versus the drug concentration.

### Example 20

### Comparative Potency of (+)3′-thia-2′,3′-dideoxycytidine, (−)3′-thia-2′,3′-dideoxycytidine and (±)3′-thia-2′,3′-dideoxycytidine as Inhibitors of HBV In Vitro

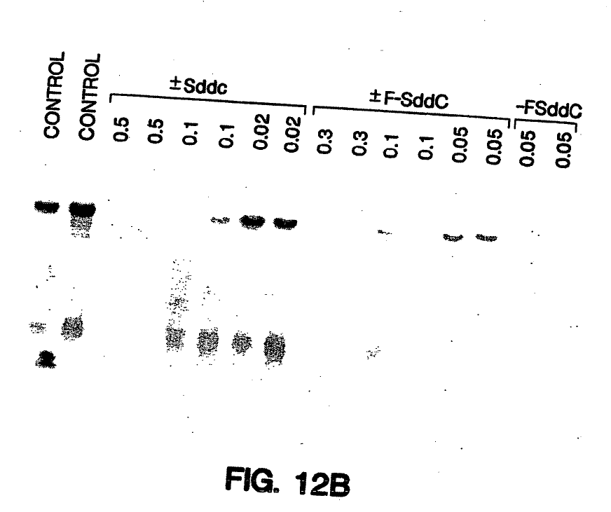

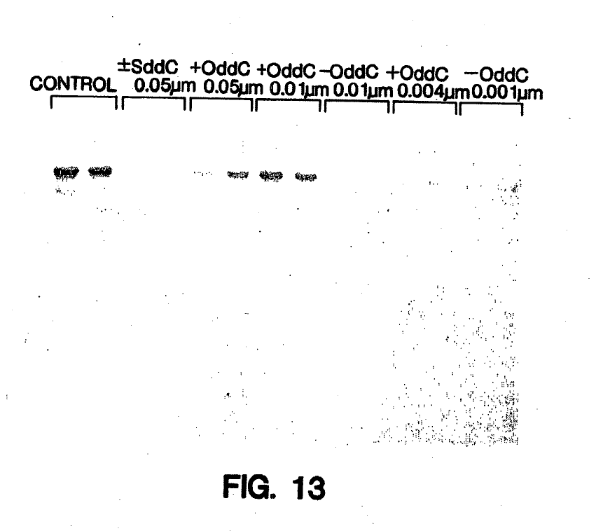

The 2.2.15 cell line was used to evaluate the antiviral activities of (+)3′-thia-2′,3′-dideoxycytidine, (−)3′-thia-2′,3′-dideoxycytidine and (±)3′-thia-2′,3′-dideoxycytidine. The antiviral effects were measured by analysis of extracellular HBV DNA (FIG. 12). The experiment revealed that the amount of extracellular HBV DNA decreased in a dose dependent manner. At a concentration of 0.5 μm (±)3′-thia-2′,3′-dideoxycytidine completely inhibited the replication of HBV, whereas there was approximately only 50% inhibition-by (+)3′-thia-2′,3′-dideoxycytidine. The HBID₅₀ of (−)3′-thia-2′,3′-dideoxycytidine was estimated to be 0.02 μm which is significantly lower than that of (±)3′-thia-2′,3′-dideoxycytidine (HBID₅₀=0.1 μm).

### Summary of the Biological Activity

The anti-HBV effect of each of the two racemic 3′-thia-2′,3′-dideoxycytidine was examined as described above. The ID₅₀ of (+)3′-thia-2′,3′-dideoxycytidine, (±)3′-thia-2′,3′-dideoxycytidine and (−)3′-thia-2′,3′-dideoxycytidine are approximately>0.5 μm, 0.1 μm and 0.02 μm, respectively, which indicates that (−)3′-thia-2′,3′-dideoxycytidine is the primary form responsible for the anti-HBV effect observed for (±)3′-thia-2′,3′-dideoxycytidine. Furthermore, the (+)-form may even interfere with the (−)-form based on the observation that the (−)-form is approximately 5 fold more active than the (±) form of 3′-thia-2′,3′-dideoxycytidine at the same dose (if there is no interference, it should be only 2-fold). A similar observation was also made for 5-fluoro-3′-thia-2′,3′-dideoxycytidine.

### Example 21

### CEM Growth in 3′-thia-2′,3′-dideoxycytidine CEM were seeded at 3.5×10⁴ cells per ml onto Corning 6-well dishes at 5 ml per well in RPMI 1640+5% dialized FBS+kanamycin at 100 mg/ml.

Two wells were treated for each of the following conditions:
3, 10, 30, 100 μm+SddC
3, 10, 30, 100 μm±SddC.

Six wells were seeded as controls. These wells were untreated.

Cell number was determined on days 2, 4 and 6 after seeding. The cells were pipetted to break clumps, and then 1 ml was removed from each well and diluted into 9 ml saline. The saline+cells were pipetted well to break clumps and counted via a coutler counter.

To determine ID₅₀, cell number was plotted versus days in culture. (+)3′-thia-2′,3′-dideoxycytidine was seen to be very toxic to the cells with an ID₅₀ of 1.26 μm. (±)3′-thia-2′,3′-dideoxycytidine was less toxic with an ID₅₀ of 7.2 μm.

The cytotoxicity studies using CEM cells and dialyzed serum showed that. (+)3′-thia-2′,3′-dideoxycytidine is more toxic (ID₅₀: 1.3 μm) than (±)3′-thia-2′,3′-dideoxycytidine (ID₅₀: 7.2 μm) suggesting that the (−)-form is less toxic than (+)-form and the (±)-form.

### Examples 22-26

The numbers in parentheses in Examples 22-26 refer to the compound numbers in Scheme 1.

### Example 22

### (−)-1,6-Anhydro-α-L-gulopyranose (3)

A mixture of (2) (33 g, 0.127 mol) and 0.5 N HCl (330 mL, 0.165 mol) was refluxed for 20 hours and the mixture was cooled and neutralized to pH 6 by a resin (Dowex-2, HCO₃-form) with air bubbling. The resin was recycled by washing with 10% HCl, water, methanol, water and saturated NAHCO₃ solution. The reaction mixture was filtered and the resin was washed with water (500 mL). The combined filtrate was concentrated to dryness and dried in vacuo overnight. The residue was purified over a column (5 cm depth, silica gel, mesh, CHCl₃-MeOH, 10:1) to give a slightly yellow solid, which was recrystallized from absolute alcohol to give a colorless solid (3) [R_{f}=0.43 (CHCl₃-MeOH, 5:1), 7.3 g, 35.52%]. The L-gulose (R_{f}=0.07, 11 g) obtained was recycled to give (3) (5 g, total yield 60%): mp 142.5-145° C.; ¹H NMR(DMSO-d₆) δ 3.22-3.68(m, 4H, H-2, -3, -4 and -6a), 3.83 (d, J_{6b,6a}=7.25 Hz, 1H, H_{b}−6), 4.22 (pseudo t, J_{5,6e}=4.61 and 4.18 Hz, H, H−5), 4.46 (d, J_{2-OH,2}=6.59 Hz, 1 H, 2-OH, exchangeable with D₂O), 4.62 (d, J_{3-OH,3}=5.28 Hz, 1 H, 3-OH, exchangeable with D₂O), 5.07 (d, J₄-OH,4=4.84 Hz, 1H, 4-OH, exchangeable with D₂O), 5.20 (d, J_{1,2}=2.19 Hz, 1H, H-1). [αa]_{d}²⁵−50.011 (c, 1.61, MeOH).

### Example 23

### (−)-(1′S, 2S,4S)-4-(1,2-Dihydroxyethyl-1,2-0-Isopropylidene)-2-hydroxymethyl)-dioxolane (5)

To a solution of (3) (11.3 g, 0.07 mol) in methanol (350 mL) was added dropwise a solution of NaIO₄ (22.36 g, 0.1 mol) in water (300 mL) for 10 minutes at 0° C. and the mixture was stirred mechanically for 15 minutes. NaBH₄ (7.91 g, 0.21 mol) was added to this mixture and the reaction mixture was stirred for 10 minutes at 0° C. The white solid was filtered off and the solid was washed with methanol (300 mL). The combined filtrate was neutralized by 0.5 N HCl (⁻200 mL) and concentrated to dryness. The residue was dried in vacuo overnight. The syrupy residue was triturated with methanol-acetone (1:5, 1200 mL) using a mechanical stirrer (5 hours) and the white solid (1st.) was filtered off. The filtrate was concentrated to dryness and the residue was dissolved in acetone (500 mL) and followed by p-TsOH (6.63 g, 0.035 mol). After stirring for 6 hours, the mixture was neutralized by Et₃N and the solid (2nd.) was filtered off and the filtrate was concentrated to dryness. The residue was dissolved in ethyl acetate (350 mL) and washed with water (500 mL×2), dried (MgSO₄), filtered, evaporated to give crude (5) (3.6 g) as a yellowish syrup. The water layer was concentrated to dryness and dried in vacuo. The solid obtained (1st and 2nd) was combined with the dried water layer and recycled using 10% methanol-acetone (900 mL), p-TsOH (16 g, 0.084 mol) by 1 hour stirring to yield crude (5) (5.6g). The crude product obtained was purified by a dry column over silica gel (MeOH—CHCl₃, 1%-5%) to give (5)(R_{f}=0.82(CHCl₃-MeOH, 10:1), 8.8 g, 61.84%] as a colorless oil. ¹H NMR (DMSO-d₆) δ 1.26 and 1.32 (2×s, 2×3 H, isopropylidene), 3.41 (dd, J_{CH2OH,OH}=6.04 Hz, J_{CH2OH,2}=3.96 Hz, 2H, CH₂OH), 3.56-4.16(m, 6H, H-4, -5, -1′ and -2′), 4.82 (t, J_{OH,CH2}=6.0 Hz, 1 H, CH₂OH, exchangeable with D₂O), 4.85 (t, J_{20H,CH20H}=3.96 Hz, 1H, H-2).[α]_{D}²⁵−12.48 (c, 1.11, CHCl₃), Anal, Calcd for C₉H₁₆O₅:C,52.93; H,7.90. Found:C,52.95; H,7.86.

### Example 24

### (+)(1′S,2S,4S)-4-(1,2-Dihydroxymethyl-1,2-O-Isopropylidene)-2-(O-benzoyloxymethyl)-dioxolane (6)

To a solution of (5) (8.5 g, 0.042 mol) in pyridine-CH₂Cl₂ (1:2, 120 mL) was added dropwise benzoyl chloride (6.5 mL, 0.056 mol) at 0° C. and the temperature was raised to room temperature. After stirring for 2 hours, the reaction was quenched with methanol (10 mL) and the mixture was concentrated to dryness In vacuo. The residue was dissolved in CH₂Cl₂ (300 mL) and washed with water (100 mL×2), brine, dried (MgSO₄), filtered, evaporated to give a yellowish syrup, which was purified by silica gel column chromatography (EtOAc-Hx, 4% -30%) to yield (6) [R_{f}=0.45 (Hx-EtOAc, 3:1), 10.7 g, 83.4%] as a colorless oil. 1H NMR (CDCl₃) δ 1.35 and 1.44 (2×s, 2×3H, isopropylidene) 3.3-4.35 (m 6H, H-4, -5, -1′ and -2′), 4.44 (d, J=3.96 Hz, 2H, CH₂—OBz), 5.29 (t, J=3.74 Hz, 1H, H-2), 7.3-7.64, 8.02-8.18 (m, 3H, 2H, —OBz).[α]_{D}²⁵+10.73(c,1.75,MeOH). Anal. Calcd for C₁₆H₂₀O₆: C, 62.33; H, 6.54. Found: C, 62.39; H, 6.54.

### Example 25

### (+)-(1′S,2S,4S)-4-(1,2-Dihydroxyethyl)-2-(O-benzoyloxymethyl)-dioxolane (7)

The mixture of (6) (5.7 g. 0.018 mol) and p-TsOH (1.05 g. 0.0055 mol) in methanol (70 mL) was stirred at room temperature for 2 hours. But the reaction was not completed, so the solvent was evaporated to half volume and were added methanol (50 mL) and p-TsOH (0.7 g, 3.68 mmol). After stirring for one more hour, the reaction mixture was neutralized by EtN and the solvent was evaporated to dryness. The residue was purified by silica gel column chromatography (Hx-EtOAC, 10%-33%) to give (7) [R_{f}=0.15(Hx-EtOAc, 1:1), 4.92 g, 99.2%] as a colorless syrup ¹H NMR (DMSO-d₆) δ 3.43 (m, 2H, H-2′), 3.67-4.1 (m, 4H, H-4, -5 and -1′), 4.32 (d, J=3.73 Hz, 2H, CH₂—OBz), 4.60 (t, J=5.72 Hz, 2′-OH, exchangeable with D₂O), 5.23 (t, J=3.96 Hz, 1H, H-2), 7.45-7.7, 7.93-8.04 (m, 3H, 2H, —OBz), [α]_{D}²⁵+9.16 (c,1.01, CHCl₃). Anal. Calcd for C₁₃H₁₆O₆: C,58.20; H,6.01. Found: C,58.02; H.6.04.

### Example 26

### (−)-(2S,4S) and (2S,4R)-4-Acetoxy-2-(6-benzoyloxymethyl)-dioxolane (9)

To a solution of (7) (3.04 g, 0.011 mol) in CCl₄—CH₃CN (1:1, 160 mL) was added a solution of NaIO₄ (10.18 g, 0.048 mol) in water (120 mL) and then RuO₂.hydrate (0.02 g). After the reaction mixture was stirred for 5 hours, the solid was removed by filtration over Celite and the filtrate was evaporated to ⅓ volume. The residue was dissolved in CCl₂(100 mL) and the water layer was extracted with CH₂Cl₂ (100 mL×2). The combined organic layer was washed with brine (50 mL), dried (MgSO₄), filtered, evaporated to dryness and dried in vacuo for 16 hours to give crude (8) (2.6 g, 91%).

To a solution of crude (8) (2.6 , 0.01 mol) in dry THF (60 mL) were added Pb(OAc)₄(5.48 g, 0.0124 mol) and pyridine (0.83 mL, 0.0103 mol) under N₂ atmosphere. The mixture was stirred for 45 minutes under N₂ and the solid was removed by filtration. The solid was washed with ethyl acetate (60 mL) and the combined organic layer was evaporated to dryness. The residue was purified by silica gel column chromatography (Hx-EtOAc, 2:1) to yield (9) [R_{f}=0.73 and 0.79 (Hx-EtOAc, 2:1), 1.9 g, 69.34%] as a colorless oil. ¹H NMR (CDCl₃) δ 1.998, 2.11 (2×s, 3H, —OAc), 3.93-4.33 (m, 2H, H-5), 4.43, 4.48 (2×d, J=3.73, 3.74 Hz, 2H, CH₂OBz), 5.46, 5.55 (2×t, J=4.18, 3.63 Hz, 1H, H-2); 6.42 (m, 1H, H-4), 7.33-7.59, 8.00-8.15 (m, 3H, 2H, —OBz).[α]_{D}²⁵−12.53 (c, 1.11, CHCl₃). Anal. Calcd for C₁₃H₁₄O₆; C, 58.64; H, 5.30. Found: C, 58.78; H, 5.34.

### Examples 27-29

The numbers in parentheses in Examples 27-29 refer to compound numbers in Scheme 2.

### Example 27

### (−)-(2S,4S)-1-[2-(benzoyl)-1,3-dioxolan-4-yl]cytosine(2), (+)-(2S,4R)-1-[2-(benzyloxy)-1,3-dioxolan-4-yl)cytosine (3)

A mixture of N⁴-acetylcytosine (1.24 g, 7.52 mmol) in dry dichloroethane (20 mL), hexamethyldisilazane (15 mL), and ammonium sulfate (cat.amount) was refluxed for 4 hours under a nitrogen atmosphere. The resulting clear solution was cooled to room temperature. To this silylated acetylcytosine a solution of (1) (1.0 g, 3.76 mmol) in dry dichloroethene (10 mL) and TMSOTf (1.46 mL 7.55 mmol) were added and stirred for 6 hours, then saturated NaHCO₃ (10 mL) was added and stirred for another 15 minutes and filtered through a Celite pad. The filtrate was evaporated and the solid was dissolved in EtOAc and washed with water and brine, dried, filtered and evaporated to give the crude product. This crude product was purified on a silica column (5% MeOH/CHCl₃) to yield a pure α,β mixture of (2) and (3) (0.40 g, 30%) and the α,β mixture of (4) and (5) (0.48 g, 40%). The mixture of (4) and (5), was reacetylated for separation, the combined α,β mixture was separated by a long silica column (3% MeOH/CHCl₃) to yield (2) (0.414 g, 30.7%) and (3) (0.481 g, 35.6%) as foams. These foams were triturated with MeOH to obtain white solids. 2: UV (MeOH) λ max 298 nm; Anal. (C₁₇H₁₇ N₃O₈) C, H, N. 3: UV (MeOH) λ max 298 nm.

### Example 28

### (−)-(2S,4S)-1-(2-Hydroxymethyl-1,3-dioxolan-4-yl)cytosine(6)

A solution of (2) (0.29 g, 0.827) in MeOH/NH₃ (50 mL, saturated at 0° C.) was stirred at room temperature for 10 hours. The solvent was evaporated and the crude was purified on preparative silica plates (20% MeOH/CHCl₃) to give an oil. This was crystallized from CCl₂/hexane to give (6) (0.136 g, 77.7%) as a white solid. UV λ max 278.0 nm (ε 11967) (pH 2), 270.0 nm (ε 774) (pH 7), 269.0 nm (ε8379) (pH 11); Anal. (C8H₁₁N₃O₄)C, H, N.

### Example 29

### (+)-(2S,4R)-1-(2-Hydroxymethyl-1,3-dioxolan-4-yl)cytosine(7)

A solution of (2) (0.35 g, 0.991) in MeOH/N; (50 mL), saturated at 0° C. was stirred at room temperature for 10 hours. The solvent was evaporated and the crude was purified on preparative silica plates (20% MeOH/CHCl₃) to give an oil. This was crystallized from CH₂Cl₂/hexane to give (7) (0.135 g, 64.3%) as a white solid. UV λ max 278.0 nm (ε 13241) pH 2), 270.0 nm (ε 13241) (pH 2), 270.0 nm (ε 8780) (pH 7), 269.0 nm (ε 9071) (pH 11); Anal. (C₈H₁₁N₃O₄) C, H, N.

### Example 30

### Results of Biological Testing

Compounds -OddC and -SddC were tested following the same procedure as in Example 20 using the 2.2.15 cell line. Compound HBVID₅₀ Toxicity (Anti CEM cell ID₅₀)

| | | |
|---|---|---|
| 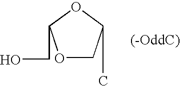 | <0.001 μM | >5 μM |
| 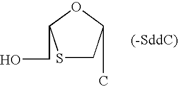 | 0.001 μM | −50 μM |

As can be seen the therapeutic index for -OddC should be equal or better than -SddC, in fact, from an economical point of view, -OddC may be even more valuable than -SddC due to its low ID₅₀ (anti HBV).

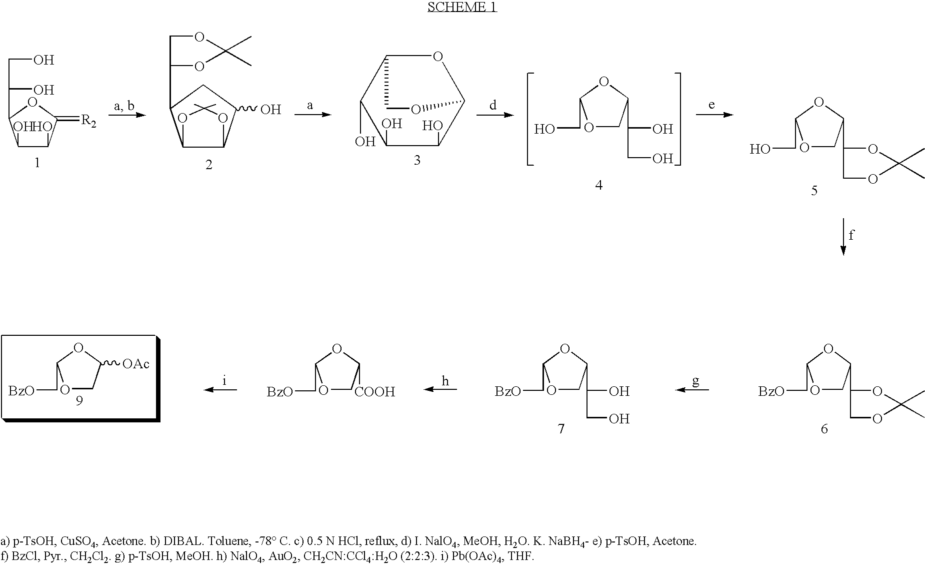

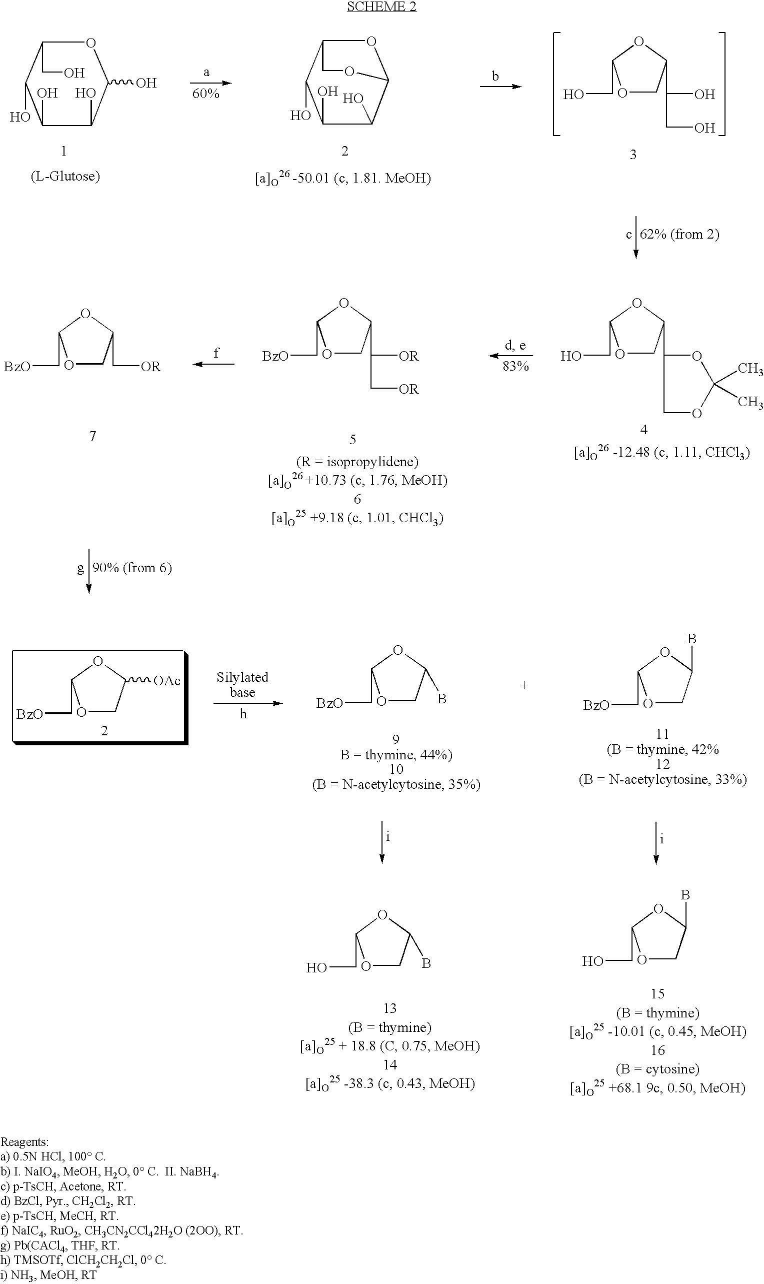

It will be appreciated that the instant specification is set forth by way of illustration, and that various modifications and changes may be made without departing from the spirit and scope of the present invention.

1. A method for treating a hepatitis B virus infection, comprising administering to a patient in need thereof an effective amount of (−)-L-β-dioxolane-cytosine or a pharmaceutically acceptable salt or ester thereof.

2. The method of claim 1, wherein the (−)-L-β-dioxolane-cytosine is administered in a dose of 1 to 100 mg/kg of body weight per day and said patient is a human.

3. The method of claim 1, wherein the (−)-L-β-dioxolane-cytosine is administered in a dose of 2 to 50 mg/kg of body weight per day and said patient is a human.

4. The method of claim 1, wherein the (−)-L-β-dioxolane-cytosine is administered in a dose of 2 to 10 mg/kg of body weight per day and said patient is a human.

5. The method of claim 1, further comprising administering an anti-viral effective amount of adenine arabinoside or interferon α.
